(19) United States
(12) Patent Application Publication
(10) Pub. No.: US 2025/0073186 A1
(43) Pub. Date: Mar. 6, 2025

(54) COMBINATION TREATMENT METHODS

(71) Applicant: Rose Research Center, LLC, Raleigh, NC (US)

(72) Inventor: Jed E. ROSE, Durham, NC (US)

(21) Appl. No.: 18/559,872

(22) PCT Filed: May 10, 2022
(86) PCT No.: PCT/US2022/028504

§ 371 Date: Nov. 9, 2023

Related U.S. Application Data
(60) Provisional application No. 63/187,001, filed on May 11, 2021.

(51) Int. Cl. A61K 31/137; A24B 15/167; A24B 15/32; A24F 40/10; A61K 31/423; A61K 31/465; A61P 25/34

(52) U.S. Cl. CPC A61K 31/137; A24B 15/167; A24B 15/32; A24F 40/10; A61K 31/423; A61K 31/465; A61P 25/34

(57) ABSTRACT

Provided herein are methods of combined treatments using bupropion and/or its metabolites and zonisamide or other similar anticonvulsants or GABAergic agents, optionally in further combination with one or more nicotine replacement or substitution products, for promoting smoking cessation, reducing craving for combustible tobacco products, treating dependency, addiction, or withdrawal associated with combustible tobacco products, and/or for facilitating a smoker to switch from combustible tobacco products to a nicotine replacement or substitution product such as e-cigarettes.

### CROSS REFERENCE TO RELATED APPLICATIONS

This application claims the benefit of priority to U.S. Provisional Application No. 63/187,001, filed May 11, 2021, the content of which is incorporated herein by reference in its entirety.

### BACKGROUND

### Field of the Disclosure

In various embodiments, the present disclosure generally relates to methods for promoting smoking cessation and/or facilitating the switch from smoking combustible tobacco products to a nicotine replacement or substitution product.

### Background

Smoking continues to be a major health hazard in our society. It is thought to be the leading preventable cause of death in the United States, resulting in over 500,000 deaths per year due to smoking-related diseases. According to the U.S. Surgeon General (2010 Report), the major smoking-related diseases, including cancer, heart and lung disease, have been linked to inhaling the combustion products of burning tobacco, rather than to nicotine per se. Moreover, smoking not only affects the health of a smoker, it also poses a health risk for non-smokers. Thus, smoking cessation or facilitating the smoker to switch to a non-combustible nicotine product is of great public interest. Despite various developments made in this field, quitting smoking remains challenging.

### BRIEF SUMMARY

In various embodiments, the present disclosure provides novel treatment methods for promoting smoking cessation and/or facilitating the switch from smoking combustible tobacco products to one or more nicotine replacement or substitution products as described herein. In a broad aspect, the present disclosure provides methods of combined treatments using bupropion and/or its metabolites and zonisamide or other similar anticonvulsants or GABAergic agents, optionally in further combination with one or more nicotine replacement or substitution products, for promoting smoking cessation, reducing craving for combustible tobacco products, and/or enhancing the aversive effects of combustible tobacco products, treating dependency, addiction, or withdrawal associated with combustible tobacco products, and/or for facilitating a smoker to switch from combustible tobacco products to a nicotine replacement or substitution product such as e-cigarettes.

The present disclosure is based in part on the discovery that combined treatments of smokers with bupropion and zonisamide facilitated smokers to switch from smoking combustible cigarettes to nicotine replacement or substitution products (e-cigarettes in the Examples) and helped smokers achieve and maintain abstinence from smoking combustible cigarettes. The present disclosure shows that the smoking cessation effects achieved from the combined treatments can be maintained for an extended period of time—the clinical results show that approximately ⅓ of participants maintained smoking abstinence between week 8 and week 11 after the target switching date. In addition, it was found that the administration of zonisamide appeared to reduce incidences of adverse events associated with bupropion, such as insomnia, which offers advantages for dosing schedules and is expected to result in better compliance. In view of these clinical results, the methods described herein can offer many advantages over existing methods for promoting smoking cessation and/or for facilitating the switch from combustible tobacco products to a nicotine replacement or substitution product such as e-cigarettes. Certain aspects of the clinical results have also been published, see Drug and Alcohol Dependence, 234:109346 (2022), the content of which is herein incorporated by reference in its entirety.

In some embodiments, the present disclosure provides:
[1] A method of treatment for promoting smoking cessation in a subject, the method comprising administering to the subject: a) bupropion or hydroxybupropion; b) an anticonvulsant or a GABAergic agent, and optionally c) a nicotine replacement or substitution product.
[2] The method of [1], comprising administering to the subject a daily dose of bupropion ranging from about 150 mg to about 450 mg, e.g., about 300 mg to about 450 mg.
[3] The method of [2], wherein the bupropion is administered in the form of a modified release formulation, such as a sustained release formulation or an extended release formulation, formulated for once daily or twice daily dosing.
[4] The method of [2], wherein the bupropion is administered in the form of an immediate release formulation.
[5] The method of any one of [2]-[4], wherein the bupropion is orally administered.
[6] The method of any one of [2]-[5], wherein the bupropion is administered to the subject once daily or twice daily.
[7] The method of any one of [2]-[6], wherein the bupropion is administered in the form of a pharmaceutically acceptable salt.
[8] The method of any one of [2]-[7], comprising administering the bupropion to the subject in the morning.
[9] The method of any one of [2]-[8], comprising administering the bupropion to the subject in the evening.
[10] The method of any one of [1]-[9], wherein the anticonvulsant or GABAergic agent is zonisamide.
[11] The method of [10], comprising administering zonisamide to the subject at a daily dose ranging from about 25 mg to about 400 mg, e.g., about 25 mg to about 100 mg, about 50 mg to about 100 mg, etc., preferably, the zonisamide is administered orally.
[12] The method of or [11], comprising administering zonisamide concurrently with bupropion, such as administering a dosage form comprising both zonisamide and bupropion as active ingredients, or concurrently administering separate dosage forms comprising zonisamide and bupropion, respectively.
[13] The method of or [11], comprising administering zonisamide and bupropion sequentially in any order.
[14] The method of any one of [10]-[13], wherein the zonisamide is administered in an amount effective in reducing one or more adverse effects associated with bupropion or hydroxybupropion.
[15] The method of any one of [10]-[14], wherein the zonisamide is administered in an amount effective in reducing incidences of insomnia and/or agitation without increasing incidences of somnolence or fatigue.
[16] The method of any one of [10]-[15], wherein the zonisamide is administered in an amount effective in reducing incidences of seizure.
[17] The method of any one of [10]-[16], wherein the zonisamide is administered in an amount effective in enhancing smoking cessation effects of bupropion.
[18] The method of any one of [10]-[17], which does not administer to the subject a nicotine replacement or substitution product.
[19] The method of any one of [10]-[17], comprising administering to the subject a nicotine replacement or substitution product.
[20] The method of [19], wherein the nicotine replacement or substitution product is an e-cigarette, nicotine patch, nicotine gum, nicotine lozenge, nicotine pouch, nasal spray nicotine, dissolvable nicotine product, sublingual nicotine tablet, and/or nicotine inhaler (including nicotine salt inhaler, such as Philip Morris International's Platform 3 product).
[21] The method of [19], wherein the nicotine replacement or substitution product is a modified risk tobacco product.
[22] The method of [21], wherein the modified risk tobacco product is a smokeless tobacco such as chewable tobacco or a noncombustible tobacco product such as a heat-not-burn system (e.g., Philip Morris International's IQOS).
[23] The method of any one of [1]-[22], wherein the subject is not in need of treatment of obesity.
[24] The method of any one of [1]-[23], wherein the subject is a smoker who uses both e-cigarettes and combustible cigarettes prior to the treatment.
[25] The method of any one of [1]-[24], wherein the subject smokes at least 10 commercially available cigarettes (combustible cigarettes) or equivalent per day for at least 12 months prior to the treatment.
[26] The method of any one of [1]-[25], wherein the subject has an expired air CO reading of at least 10 ppm prior to the treatment.
[27] The method of any one of [1]-[26], which reduces the subject's daily consumption of combustible cigarettes.
[28] The method of any one of [1]-[27], which reduces the subject's expired air CO level by 20% or more (e.g., about 50%) compared to baseline and/or reduces the urinary NNAL (4-(methylnitrosamino)-1-(3-pyridyl)-1-butanol) by 20% or more (e.g., about 50%) compared to baseline.
[29] The method of any one of [1]-[28], which reduces the subject's craving for combustible cigarettes, such as reduces the average per-item craving score of the subject to less than or equal to 4 on the 7 point Shiffman-Jarvik Withdrawal Scale (or less than 50 on a 100-point Visual Analog Scale) and/or the average per-item craving score of the subject does not increase by more than 1 point based on the 7 point Shiffman-Jarvik Withdrawal Scale (or by more than 10 points on a 100-point Visual Analog Scale).
[30] The method of any one of [1]-[29], wherein the subject achieves smoking abstinence from combustible cigarettes.
[31] A method of treatment for facilitating a smoker to switch from combustible cigarettes to e-cigarettes or another nicotine replacement or substitution product, the method comprising administering to the smoker: a) bupropion or hydroxybupropion; and b) an anticonvulsant or a GABAergic agent.
[32] The method of [31], comprising administering to the smoker a daily dose of bupropion ranging from about 150 mg to about 450 mg, e.g., about 300 mg to about 450 mg.
[33] The method of [32], wherein the bupropion is administered in the form of a modified release formulation, such as a sustained release formulation or an extended release formulation, formulated for once daily or twice daily dosing.
[34] The method of [33], wherein the bupropion is administered in the form of an immediate release formulation.
[35] The method of any one of [32]-[34], wherein the bupropion is orally administered.
[36] The method of any one of [32]-[35], wherein the bupropion is administered to the smoker once daily or twice daily.
[37] The method of any one of [32]-[36], wherein the bupropion is administered in the form of a pharmaceutically acceptable salt.
[38] The method of any one of [32]-[37], comprising administering the bupropion to the smoker in the morning.
[39] The method of any one of [32]-[38], comprising administering the bupropion to the smoker in the evening.
[40] The method of any one of [31]-[39], wherein the anticonvulsant or GABAergic agent is zonisamide.
[41] The method of [40], comprising administering zonisamide in a daily dose ranging from about 25 mg to about 400 mg, e.g., about 25 mg to about 100 mg, about 50 mg to about 100 mg, etc., preferably, the zonisamide is administered orally.
[42] The method of or [41], comprising administering zonisamide concurrently with bupropion, such as administering a dosage form comprising both zonisamide and bupropion as active ingredients, or concurrently administering separate dosage forms comprising zonisamide and bupropion, respectively.
[43] The method of or [41], comprising administering zonisamide and bupropion sequentially in any order.
[44] The method of any one of [40]-[43], wherein the zonisamide is administered in an amount effective in reducing one or more adverse effects associated with bupropion or hydroxybuproprion.
[45] The method of any one of [40]-[44], wherein the zonisamide is administered in an amount effective in reducing incidences of insomnia and/or agitation without increasing incidences of somnolence or fatigue.
[46] The method of any one of [40]-[45], wherein the zonisamide is administered in an amount effective in reducing incidences of seizure.
[47] The method of any one of [40]-[46], wherein the zonisamide is administered in an amount effective in enhancing smoking cessation effects of bupropion.
[48] The method of any one of [31]-[47], for facilitating the smoker to switch from combustible cigarettes to e-cigarettes.
[49] The method of any one of [31]-[47], for facilitating the smoker to switch from combustible cigarettes to a nicotine replacement or substitution product selected from an e-cigarette, nicotine patch, nicotine gum, nicotine lozenge, nicotine pouch, nasal spray nicotine, dissolvable nicotine product, sublingual nicotine tablet, nicotine inhaler (including nicotine salt inhaler, such as Philip Morris International's Platform 3 product), and a modified risk tobacco product, e.g., a smokeless tobacco such as chewable tobacco or a noncombustible tobacco product such as a heat-not-burn system (e.g., Philip Morris International's IQOS).
[50] The method of any one of [31]-[49], wherein the smoker is not in need of treatment of obesity.
[51] The method of any one of [31]-[50], wherein the smoker uses both e-cigarettes and combustible cigarettes prior to the treatment.
[52] The method of any one of [31]-[51], wherein the smoker smokes at least 10 commercially available cigarettes (combustible cigarettes) or equivalent per day for at least 12 months prior to the treatment.
[53] The method of any one of [31]-[52], wherein the smoker has an expired air CO reading of at least 10 ppm prior to the treatment.
[54] The method of any one of [31]-[53], which reduces the smoker's daily consumption of combustible cigarettes.
[55] The method of any one of [31]-[54], which reduces the smoker's expired air CO level by 20% or more (e.g., about 50%) compared to baseline and/or reduces the urinary NNAL (4-(methylnitrosamino)-1-(3-pyridyl)-1-butanol) by 20% or more (e.g., about 50%) compared to baseline.
[56] The method of any one of [31]-[55], which reduces the smoker's craving for combustible cigarettes, such as reduces the average per-item craving score of the subject to less than or equal to 4 on the 7 point Shiffman-Jarvik Withdrawal Scale (or less than 50 on a 100-point Visual Analog Scale) and/or the average per-item craving score of the subject does not increase by more than 1 point based on the 7 point Shiffman-Jarvik Withdrawal Scale (or by more than 10 points on a 100-point Visual Analog Scale).
[57] The method of any one of [31]-[56], wherein the smoker achieves smoking abstinence from combustible cigarettes.
[58] A method of treatment for reducing a subject's craving for combustible cigarettes, comprising administering to the subject: a) bupropion or hydroxybupropion; b) an anticonvulsant or a GABAergic agent, and optionally c) a nicotine replacement or substitution product.
[59] The method of [58], comprising administering to the subject a daily dose of bupropion ranging from about 150 mg to about 450 mg, e.g., about 300 mg to about 450 mg.
[60] The method of [59], wherein the bupropion is administered in the form of a modified release formulation, such as a sustained release formulation or an extended release formulation, formulated for once daily or twice daily dosing.
[61] The method of [59], wherein the bupropion is administered in the form of an immediate release formulation.
[62] The method of any one of [59]-[61], wherein the bupropion is orally administered.
[63] The method of any one of [59]-[62], wherein the bupropion is administered to the subject once daily or twice daily.
[64] The method of any one of [59]-[63], wherein the bupropion is administered in the form of a pharmaceutically acceptable salt.
[65] The method of any one of [59]-[64], comprising administering the bupropion to the subject in the morning.
[66] The method of any one of [59]-[65], comprising administering the bupropion to the subject in the evening.
[67] The method of any one of [58]-[66], wherein the anticonvulsant or GABAergic agent is zonisamide.
[68] The method of [67], comprising administering zonisamide to the subject at a daily dose ranging from about 25 mg to about 400 mg, e.g., about 25 mg to about 100 mg, about 50 mg to about 100 mg, etc., preferably, the zonisamide is administered orally.
[69] The method of or [68], comprising administering zonisamide concurrently with bupropion, such as administering a dosage form comprising both zonisamide and bupropion as active ingredients, or concurrently administering separate dosage forms comprising zonisamide and bupropion, respectively.
[70] The method of or [68], comprising administering zonisamide and bupropion sequentially in any order.
[71] The method of any one of [67]-[70], wherein the zonisamide is administered in an amount effective in reducing one or more adverse effects associated with bupropion or hydroxybupropion.
[72] The method of any one of [67]-[71], wherein the zonisamide is administered in an amount effective in reducing incidences of insomnia and/or agitation without increasing incidences of somnolence or fatigue.
[73] The method of any one of [67]-[72], wherein the zonisamide is administered in an amount effective in reducing incidences of seizure.
[74] The method of any one of [67]-[73], wherein the zonisamide is administered in an amount effective in enhancing smoking cessation effects of bupropion.
[75] The method of any one of [67]-[74], which does not administer to the subject a nicotine replacement or substitution product.
[76] The method of any one of [67]-[74], comprising administering to the subject a nicotine replacement or substitution product.
[77] The method of [76], wherein the nicotine replacement or substitution product is an e-cigarette, nicotine patch, nicotine gum, nicotine lozenge, nicotine pouch, nasal spray nicotine, dissolvable nicotine product, sublingual nicotine tablet, and/or nicotine inhaler (including nicotine salt inhaler, such as Philip Morris International's Platform 3 product).
[78] The method of [76], wherein the nicotine replacement or substitution product is a modified risk tobacco product.
[79] The method of [78], wherein the modified risk tobacco product is a smokeless tobacco such as chewable tobacco or a noncombustible tobacco product such as a heat-not-burn system (e.g., Philip Morris International's IQOS).
[80] The method of any one of [58]-[79], wherein the subject is not in need of treatment of obesity.
[81] The method of any one of [58]-[80], wherein the subject is a smoker who uses both e-cigarettes and combustible cigarettes prior to the treatment.
[82] The method of any one of [58]-[81], wherein the subject smokes at least 10 commercially available cigarettes (combustible cigarettes) or equivalent per day for at least 12 months prior to the treatment.
[83] The method of any one of [58]-[82], wherein the subject has an expired air CO reading of at least 10 ppm prior to the treatment.
[84] The method of any one of [58]-[83], which reduces the subject's daily consumption of combustible cigarettes.
[85] The method of any one of [58]-[84], which reduces the subject's expired air CO level by 20% or more (e.g., about 50%) compared to baseline and/or reduces the urinary NNAL (4-(methylnitrosamino)-1-(3-pyridyl)-1-butanol) by 20% or more (e.g., about 50%) compared to baseline.
[86] The method of any one of [58]-[85], which reduces the average per-item craving score of the subject to less than or equal to 4 on the 7 point Shiffman-Jarvik Withdrawal Scale (or less than 50 on a 100-point Visual Analog Scale).
[87] The method of any one of [58]-[86], wherein the average per-item craving score of the subject does not increase by more than 1 point based on the 7 point Shiffman-Jarvik Withdrawal Scale (or by more than 10 points on a 100-point Visual Analog Scale).
[88] The method of any one of [58]-[87], wherein the subject achieves smoking abstinence from combustible cigarettes.
[89] A method of treating dependency, addiction, or withdrawal associated with combustible cigarettes in a subject, the method comprising administering to the subject: a) bupropion or hydroxybupropion; b) an anticonvulsant or a GABAergic agent, and optionally c) a nicotine replacement or substitution product.
[90] The method of [89], comprising administering to the subject a daily dose of bupropion ranging from about 150 mg to about 450 mg, e.g., about 300 mg to about 450 mg.
[91] The method of [90], wherein the bupropion is administered in the form of a modified release formulation, such as a sustained release formulation or an extended release formulation, formulated for once daily or twice daily dosing.
[92] The method of [90], wherein the bupropion is administered in the form of an immediate release formulation.
[93] The method of any one of [90]-[92], wherein the bupropion is orally administered.
[94] The method of any one of [90]-[93], wherein the bupropion is administered to the subject once daily or twice daily.
[95] The method of any one of [90]-[94], wherein the bupropion is administered in the form of a pharmaceutically acceptable salt.
[96] The method of any one of [90]-[95], comprising administering the bupropion to the subject in the morning.
[97] The method of any one of [90]-[96], comprising administering the bupropion to the subject in the evening.
[98] The method of any one of [89]-[97], wherein the anticonvulsant or GABAergic agent is zonisamide.
[99] The method of [98], comprising administering zonisamide to the subject at a daily dose ranging from about 25 mg to about 400 mg, e.g., about 25 mg to about 100 mg, about 50 mg to about 100 mg, etc., preferably, the zonisamide is administered orally.
[100] The method of or [99], comprising administering zonisamide concurrently with bupropion, such as administering a dosage form comprising both zonisamide and bupropion as active ingredients, or concurrently administering separate dosage forms comprising zonisamide and bupropion, respectively.
[101] The method of or [99], comprising administering zonisamide and bupropion sequentially in any order.
[102] The method of any one of [98]-[101], wherein the zonisamide is administered in an amount effective in reducing one or more adverse effects associated with bupropion or hydroxybupropion.
[103] The method of any one of [98]-[102], wherein the zonisamide is administered in an amount effective in reducing incidences of insomnia and/or agitation without increasing incidences of somnolence or fatigue.
[104] The method of any one of [98]-[103], wherein the zonisamide is administered in an amount effective in reducing incidences of seizure.
[105] The method of any one of [98]-[104], wherein the zonisamide is administered in an amount effective in enhancing smoking cessation effects of bupropion.
[106] The method of any one of [98]-[105], which does not administer to the subject a nicotine replacement or substitution product.
[107] The method of any one of [98]-[105], comprising administering to the subject a nicotine replacement or substitution product.
[108] The method of [107], wherein the nicotine replacement or substitution product is an e-cigarette, nicotine patch, nicotine gum, nicotine lozenge, nicotine pouch, nasal spray nicotine, dissolvable nicotine product, sublingual nicotine tablet, and/or nicotine inhaler (including nicotine salt inhaler, such as Philip Morris International's Platform 3 product).
[109] The method of [107], wherein the nicotine replacement or substitution product is a
[110] The method of [109], wherein the modified risk tobacco product is a smokeless tobacco such as chewable tobacco or a noncombustible tobacco product such as a heat-not-burn system (e.g., Philip Morris International's IQOS).
[111] The method of any one of [89]-[110], wherein the subject is not in need of treatment of obesity.
[112] The method of any one of [89]-[111], wherein the subject is a smoker who uses both e-cigarettes and combustible cigarettes prior to the treatment.
[113] The method of any one of [89]-[112], wherein the subject smokes at least 10 commercially available cigarettes (combustible cigarettes) or equivalent per day for at least 12 months prior to the treatment.
[114] The method of any one of [89]-[113], wherein the subject has an expired air CO reading of at least 10 ppm prior to the treatment.
[115] The method of any one of [89]-[114], which reduces the subject's daily consumption of combustible cigarettes.
[116] The method of any one of [89]-[115], which reduces the subject's expired air CO level by 20% or more (e.g., about 50%) compared to baseline and/or reduces the urinary NNAL (4-(methylnitrosamino)-1-(3-pyridyl)-1-butanol) by 20% or more (e.g., about 50%) compared to baseline.
[117] The method of any one of [89]-[116], which reduces the subject's craving for combustible cigarettes, such as reduces the average per-item craving score of the subject to less than or equal to 4 on the 7 point Shiffman-Jarvik Withdrawal Scale (or less than 50 on a 100-point Visual Analog Scale) and/or the average per-item craving score of the subject does not increase by more than 1 point based on the 7 point Shiffman-Jarvik Withdrawal Scale (or by more than 10 points on a 100-point Visual Analog Scale).
[118] The method of any one of [89]-[117], wherein the subject achieves smoking abstinence from combustible cigarettes.
[119] A method of reducing the rewarding effects of smoking (e.g., satisfaction, enjoyment of the taste, enhanced attention, reduced irritability) and/or increasing the aversive effects of smoking (e.g. nausea, dizziness) of combustible tobacco products, such as combustible cigarettes, in a subject, the method comprising administering to the subject: a) bupropion or hydroxybupropion; b) an anticonvulsant or a GABAergic agent, and optionally c) a nicotine replacement or substitution product.
[120] The method of [119], comprising administering to the subject a daily dose of bupropion ranging from about 150 mg to about 450 mg, e.g., about 300 mg to about 450 mg.
[121] The method of [120], wherein the bupropion is administered in the form of a modified release formulation, such as a sustained release formulation or an extended release formulation, formulated for once daily or twice daily dosing.
[122] The method of [120], wherein the bupropion is administered in the form of an immediate release formulation.
[123] The method of any one of [120]-[122], wherein the bupropion is orally administered.
[124] The method of any one of [120]-[123], wherein the bupropion is administered to the subject once daily or twice daily.
[125] The method of any one of [120]-[124], wherein the bupropion is administered in the form of a pharmaceutically acceptable salt.
[126] The method of any one of [120]-[125], comprising administering the bupropion to the subject in the morning.
[127] The method of any one of [120]-[126], comprising administering the bupropion to the subject in the evening.
[128] The method of any one of [119]-[127], wherein the anticonvulsant or GABAergic agent is zonisamide.
[129] The method of [128], comprising administering zonisamide to the subject at a daily dose ranging from about 25 mg to about 400 mg, e.g., about 25 mg to about 100 mg, about 50 mg to about 100 mg, etc., preferably, the zonisamide is administered orally.
[130] The method of or [129], comprising administering zonisamide concurrently with bupropion, such as administering a dosage form comprising both zonisamide and bupropion as active ingredients, or concurrently administering separate dosage forms comprising zonisamide and bupropion, respectively.
[131] The method of or [129], comprising administering zonisamide and bupropion sequentially in any order.
[132] The method of any one of [128]-[131], wherein the zonisamide is administered in an amount effective in reducing one or more adverse effects associated with bupropion or hydroxybupropion.
[133] The method of any one of [128]-[132], wherein the zonisamide is administered in an amount effective in reducing incidences of insomnia and/or agitation without increasing incidences of somnolence or fatigue.
[134] The method of any one of [128]-[133], wherein the zonisamide is administered in an amount effective in reducing incidences of seizure.
[135] The method of any one of [128]-[134], wherein the zonisamide is administered in an amount effective in enhancing smoking cessation effects of bupropion.
[136] The method of any one of [128]-[135], which does not administer to the subject a nicotine replacement or substitution product.
[137] The method of any one of [128]-[135], comprising administering to the subject a nicotine replacement or substitution product.
[138] The method of [137], wherein the nicotine replacement or substitution product is an e-cigarette, nicotine patch, nicotine gum, nicotine lozenge, nicotine pouch, nasal spray nicotine, dissolvable nicotine product, sublingual nicotine tablet, and/or nicotine inhaler (including nicotine salt inhaler, such as Philip Morris International's Platform 3 product).
[139] The method of [137], wherein the nicotine replacement or substitution product is a
[140] The method of [139], wherein the modified risk tobacco product is a smokeless tobacco such as chewable tobacco or a noncombustible tobacco product such as a heat-not-burn system (e.g., Philip Morris International's IQOS).
[141] The method of any one of [119]-[140], wherein the subject is not in need of treatment of obesity.
[142] The method of any one of [119]-[141], wherein the subject is a smoker who uses both e-cigarettes and combustible cigarettes prior to the treatment.
[143] The method of any one of [119]-[142], wherein the subject smokes at least 10 commercially available cigarettes (combustible cigarettes) or equivalent per day for at least 12 months prior to the treatment.
[144] The method of any one of [119]-[143], wherein the subject has an expired air CO reading of at least 10 ppm prior to the treatment.
[145] The method of any one of [119]-[144], which reduces the subject's daily consumption of combustible cigarettes.
[146] The method of any one of [119]-[145], which reduces the subject's expired air CO level by 20% or more (e.g., about 50%) compared to baseline and/or reduces the urinary NNAL (4-(methylnitrosamino)-1-(3-pyridyl)-1-butanol) by 20% or more (e.g., about 50%) compared to baseline.
[147] The method of any one of [119]-[146], which reduces the subject's craving for combustible cigarettes, such as reduces the average per-item craving score of the subject to less than or equal to 4 on the 7 point Shiffman-Jarvik Withdrawal Scale (or less than 50 on a 100-point Visual Analog Scale) and/or the average per-item craving score of the subject does not increase by more than 1 point based on the 7 point Shiffman-Jarvik Withdrawal Scale (or by more than 10 points on a 100-point Visual Analog Scale).
[148] The method of any one of [119]-[147], wherein the subject achieves smoking abstinence from combustible cigarettes.
[149] A kit comprising: a) one or more daily doses of bupropion, each comprising an extended release formulation comprising about 150 mg to about 450 mg bupropion; b) one or more daily doses of zonisamide, each comprising a pharmaceutical composition comprising about 25 mg to about 400 mg zonisamide, and optionally c) a nicotine replacement or substitution product.
[150] The kit of [149], wherein the extended release formulation is formulated for once daily dosing.

It is to be understood that both the foregoing summary and the following detailed description are exemplary and explanatory only, and are not restrictive of the invention herein.

### BRIEF DESCRIPTION OF THE FIGURES

FIG. 1 shows an overall study design of the open-label study of Example 1 exploring the impact of combination zonisamide and bupropion on the process of switching from combustible cigarettes (CC) to an Electronic Nicotine Delivery System (“ENDS”), which is e-cigarette in this Example 1.

FIG. 2 shows a graph of the mean (±standard error) expired air carbon monoxide levels (CO) in ppm, a quantitative marker of cigarette smoke inhalation, for the study participants of Example 1, during the 11 weeks after the target “switching” date, the mean data is calculated based on the same set of participants at each time point.

FIG. 3 shows the questionnaire of the Shiffman-Jarvik Craving Scale used in the study of Example 1.

FIG. 4A shows the modified Cigarette Evaluation Questionnaire used in the study of Example 1. FIG. 4B shows the modified Electronic Cigarette Evaluation Questionnaire used in the study of Example 1.

FIG. 5 presents a graph showing the changes of craving for combustible cigarettes (CC), assessed at sessions conducted over a 13-week period (assessments began after the screening session (S1), not shown). Points represent the means (±s.e.m.) of data from the same participants reporting at all sessions (N=22), with ratings of 1 (“not at all”), 2 (“very little”), 3 (“a little”), 4 (“moderately”), 5 (“a lot”), 6 (“quite a lot”), and 7 (“extremely”).

FIG. 6 presents graphs showing the changes of ratings of the rewarding/aversive effects of combustible cigarettes (CC) and ENDS, assessed at sessions conducted over a 13-week period (assessments began after the screening session (S1), not shown). Points represent the means (±s.e.m.) of data from the same participants reporting at all sessions (N=10 abstinent and N=16 non-abstinent).

### DETAILED DESCRIPTION

The present disclosure is generally directed to combined treatments using bupropion and/or its metabolites and zonisamide or other similar anticonvulsants or GABAergic agents, e.g., for promoting smoking cessation and/or facilitating switching from smoking combustible tobacco products to a nicotine replacement or substitution product.

Zonisamide is a U.S. FDA-approved medication with an anti-seizure indication, with daily doses usually ranging from 100 mg/day to 600 mg/day. See e.g., Renu Kadian; Anil Kumar, In: StatPearls. Treasure Island (FL): StatPearls Publishing; 2020. available at: www.ncbi.nlm.nih.gov/books/NBK507903. It has multiple mechanisms of action, which include inhibiting activation of voltage-gated sodium channels at therapeutic levels. In addition, zonisamide also inhibits glutamate-mediated neurotransmission and enhances inhibitory GABA-ergic as well as serotonergic neurotransmission. (See e.g., Biton V. Clin Neuropharmacol. 2007; 30(4): 230-240 and Leppik I E. Seizure. 2004; 13 Suppl 1: S5-9; discussion S10.) It also enhances dopamine levels in the striatum, which in theory could help replace the desired effects of addictive drugs such as nicotine that also raise striatal dopamine. Zonisamide has been shown to reduce ad libitum smoking as well as to relieve craving for cigarettes. It also has been shown to decrease anger, restlessness and impatience during smoking abstinence. Dunn K E, et al. Nicotine Tob Res Off J Soc Res Nicotine Tob. 2016; 18(5): 1171-1179.

Bupropion inhibits reuptake of both noradrenaline and dopamine. This medication has been approved as an antidepressant for over 20 years. The United States Food and Drug Administration (FDA) approved the use of bupropion for smoking cessation in 1997. It is only one of two non-nicotine medications currently approved by the FDA for this purpose. Wilkes S. Int J Chron Obstruct Pulmon Dis. 2008; 3(1): 45-53. The combination of bupropion and zonisamide has been studied as a treatment for weight loss in obese adults. The usual dose for bupropion for smoking cessation is 150 mg once a day for 3 days, followed by 300 mg once a day for 7 to 12 weeks. The maximum prescribed dose for bupropion is 450 mg a day, which is the maximum dose for treating major depressive disorder and attention deficit hyperactivity disorder for adults.

The zonisamide/bupropion combination has an additional rationale in that the potential side effects of each agent can be offset by the other. For example, bupropion is associated with side effects of agitation and insomnia while zonisamide has sedative properties. Conversely, the side effects of zonisamide include sedation, which are expected to be partially offset by bupropion's stimulant actions. The combination of bupropion and zonisamide has been shown to be well tolerated in studies of weight loss produced by this drug combination. Gadde K M, et al. J Clin Psychiatry. 2007; 68(8): 1226-1229. Some of the common side effects reported for Empatic treatment (bupropion SR and zonisamide SR) for weight loss were insomnia, nausea and headache. See e.g. www.tesofensine-information.com/empatic.html

### Smoking Cessation

In some embodiments, the present disclosure provides various methods of treatments related to quitting smoking combustible tobacco products, in particular, quitting smoking combustible cigarettes.

Some embodiments of the present disclosure are directed to methods for promoting smoking cessation in a subject, which comprise administering to the subject a) bupropion or hydroxybupropion; b) an anticonvulsant or a GABAergic agent, and optionally c) a nicotine replacement or substitution product. As used herein, the method for “promoting smoking cessation” should be understood as encompassing any method that helps a human to quit or reduce combustible tobacco smoking or to quit or reduce use of combustible tobacco products; to decrease craving for combustible tobacco products; to reduce relapse to heavy smoking during withdrawal or once smoking abstinence has been achieved; and/or to alleviate various symptoms of the smoking withdrawal syndrome. In some embodiments, the method for “promoting smoking cessation” also encompasses methods for reducing the rewarding effects of combustible tobacco use and/or increasing the aversive effects associated with combustible tobacco use. In any of the embodiments described herein, unless specified or otherwise contrary from context, the combustible tobacco products can be combustible cigarettes.

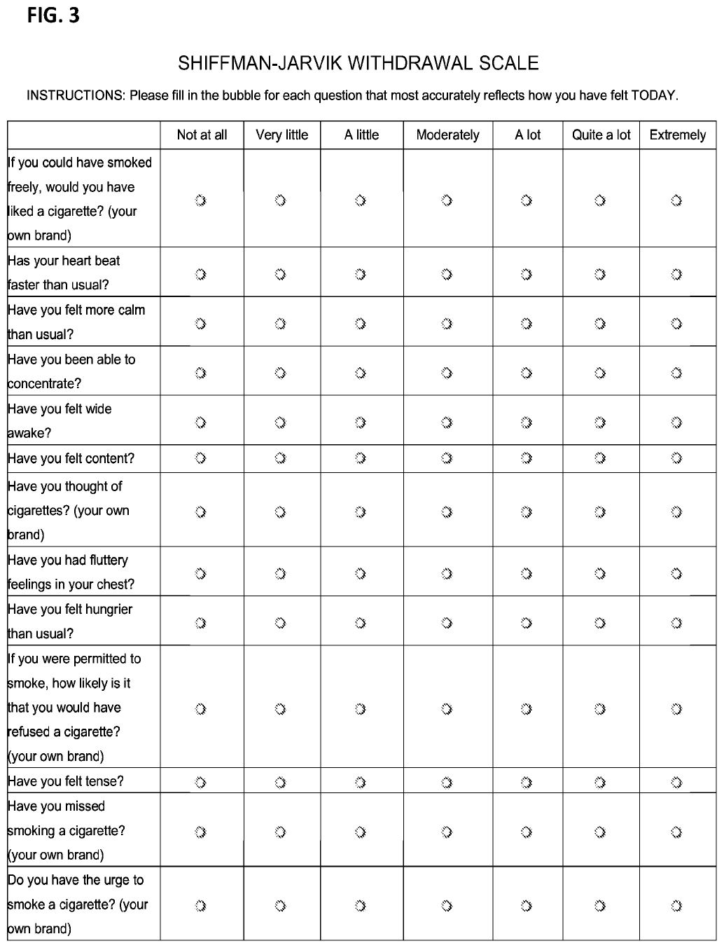

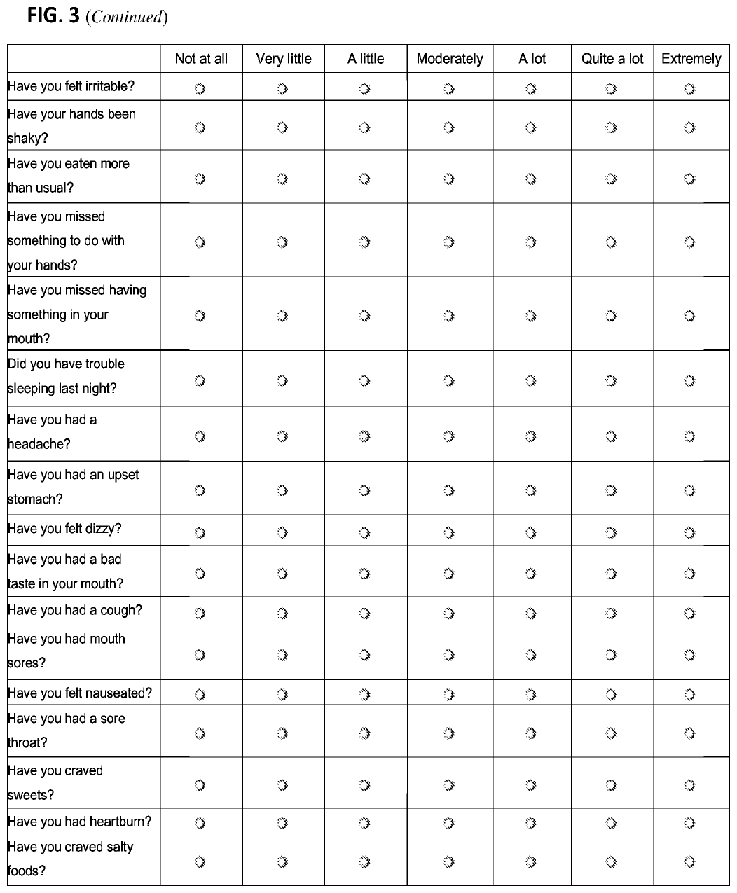

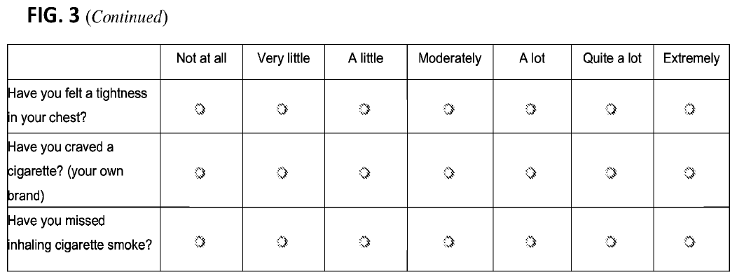

In some embodiments, the present disclosure also provides methods for reducing a subject's craving for combustible tobacco products, such as cigarettes, which comprise administering to the subject a) bupropion or hydroxybupropion; b) an anticonvulsant or a GABAergic agent, and optionally c) a nicotine replacement or substitution product. In some embodiments, reducing a subject's craving for combustible tobacco products, such as cigarettes, includes reducing the subject's average per-item craving score to be less than or equal to 4 on the 7 point Shiffman-Jarvik Withdrawal Scale (or less than 50 on a 100-point Visual Analog Scale). Typically, during the process of quitting smoking, smokers' craving scores tend to increase in comparison to baseline levels. Thus, reducing a smoker's craving also includes preventing or reducing the increase of the smoker's craving score. For example, in some embodiments, reducing a subject's craving for combustible tobacco products, such as cigarettes, includes not increasing the subject's average per-item craving score by more than 1 point based on the 7 point score on the Shiffman-Jarvik Withdrawal Scale (or by more than 10 points on a 100-point Visual Analog Scale), for example, the subject's craving score may be maintained or reduced when compared to baseline, or the subject's average per-item craving score may be increased over baseline, but by less than 1 point. The version of the Shiffman-Jarvik Withdrawal Scale used in the study described below (see FIG. 3) consists of 33 seven-point items, which are used to determine the scores for five subscales (i.e., craving, psychological, physical, stimulation/sedative, and appetite symptoms) and a total withdrawal score. The maximum score possible on each item of the five Shiffman-Jarvik Withdrawal Scale subscales is 7, and the highest possible total score for the craving scale is 42, but the maximum average per-item score is 7. In each case, a higher score indicates more severe withdrawal. To be clear, as used herein, unless otherwise specified or obviously contrary from context, the score of a subscale of the Shiffman-Jarvik Withdrawal Scale, such as the craving score, should be understood as referring to the average per-item score, with the maximum score being 7.

In some embodiments, the present disclosure further provides methods for treating dependency, addiction, or withdrawal associated with combustible tobacco products, such as cigarettes, in a subject, which comprise administering to the subject a) bupropion or hydroxybupropion; b) an anticonvulsant or a GABAergic agent, and optionally c) a nicotine replacement or substitution product.

In some embodiments, the present disclosure further provides methods for reducing the rewarding effects of smoking (e.g., satisfaction, enjoyment of the taste, enhanced attention, reduced irritability) and/or increasing the aversive effects of smoking (e.g. nausea, dizziness) of combustible tobacco products, such as combustible cigarettes, in a subject, the method comprising administering to the subject: a) bupropion or hydroxybupropion; b) an anticonvulsant or a GABAergic agent, and optionally c) a nicotine replacement or substitution product.

Various subjects are suitable to be treated with the methods herein. Typically, the subject wants to quit smoking combustible cigarettes. In some embodiments, the subject is a smoker who uses both e-cigarettes and combustible cigarettes prior to the treatment. In some embodiments, the subject smokes at least 10 commercially available cigarettes (combustible cigarettes) or equivalent per day for at least 12 months prior to the treatment. In some embodiments, the subject has an expired air CO reading of at least 10 ppm prior to the treatment. In some embodiments, the subject is not in need of treatment of obesity.

The methods herein can also be typically characterized by certain treatment effects. For example, in some embodiments, the methods herein can reduce the subject's daily consumption of combustible cigarettes, for example, compared to the daily consumption prior to the treatment, the number of combustible cigarettes is reduced by 20% or more, or 50% or more, and up to 100%. In some embodiments, the subject achieves abstinence from combustible cigarettes. In some embodiments, the methods herein can reduce the subject's expired air CO level by 20% or more (e.g., about 50%) compared to baseline (i.e., the expired air CO level prior to the treatment). For example, in some embodiments, the methods can reduce the subject's expired air CO level to be less than 5 ppm. In some embodiments, the methods herein can also reduce the subject's urinary NNAL (4-(methylnitrosamino)-1-(3-pyridyl)-1-butanol) level by 20% or more (e.g., about 50%) compared to baseline. In some embodiments, the methods herein can reduce the subject's craving for combustible cigarettes. For example, in some embodiments, the treated subject can achieve an average per-item craving score less than or equal to 4 on the 7 point Shiffman-Jarvik Withdrawal Scale (or less than 50 on a 100-point Visual Analog Scale). In some embodiments, the average per-item craving score of the treated subject is not increased by more than 1 point based on the 7 point Shiffman-Jarvik Withdrawal Scale (or by more than 10 points on a 100-point Visual Analog Scale). In any of the embodiments described herein, unless otherwise specified or contrary from context, the treatment effects described herein can be achieved and/or maintained during the treatment period and beyond, such as after 1, 2, 3, 4, 5, or 6 months of treatment. In any of the embodiments described herein, unless otherwise specified or contrary from context, the methods herein can also be characterized by reduced adverse effects associated with bupropion or hydroxybupropion. For example, in some embodiments, the methods herein can be characterized by reduced incidences of insomnia and/or agitation compared to treatment with bupropion alone. In some embodiments, the methods herein can also be characterized by reduced incidences of seizure. The treatment effects described herein can be typically achieved through using an effective dosing regimen of bupropion or hydroxybupropion and the anticonvulsant or a GABAergic agent, in combination with the optional use of the nicotine replacement or substitution product.

Typically, the methods herein comprise administering a pharmaceutical composition comprising an effective amount of bupropion to the subject. In some embodiments, the method can include administering a pharmaceutical composition comprising an effective amount of the active metabolite of bupropion, hydroxybupropion (6-hydroxybupropion), to the subject or a pharmaceutical composition comprising an effective amount of one or more prodrugs of hydroxybupropion. The bupropion useful for the methods herein is not particularly limited to any specific form. For example, bupropion in its free form or a pharmaceutically acceptable salt, such as an HCl salt, can be used in the methods herein. To be clear, the amount of bupropion, such as a daily dose referred to herein, should be understood as the equivalent amount of bupropion hydrochloride. Bupropion typically exists as a racemic mixture, such as in the marketed product under the tradename of Wellbutrin® or Zyban®. Suitable formulations of bupropion for the methods herein include any of those described herein.

The anticonvulsant or GABAergic agent that can be administered to the subject for the methods herein is also not particularly limited. However, in preferred embodiments, the anticonvulsant or GABAergic agent is zonisamide. Other anticonvulsants or GABAergic agents that act similarly to zonisamide (e.g., having a similar mechanism of action or otherwise having a similar pharmacological effect) may also be used for the methods herein. As with bupropion, the zonisamide useful for the methods herein is also not particularly limited to any specific form. For example, zonisamide in its free form or a pharmaceutically acceptable salt, such as a sodium salt, can be used in the methods herein. The amount of zonisamide, such as a daily dose referred to herein, should be understood as the equivalent amount of zonisamide in its free form. Suitable formulations of zonisamide for the methods herein include any of those described herein.

Typically, the nicotine replacement or substitution product is also administered (e.g., self-administered) to the subject in the methods herein. However, in some embodiments, the methods herein do not include administering the nicotine replacement or substitution product. When administered, suitable nicotine replacement or substitution product is not particularly limited and broadly includes any products that can deliver nicotine to a subject user in a non-combustion manner. Non-limiting nicotine replacement or substitution products useful for the methods herein include any of those described herein.

### Switching to Nicotine Replacement or Substitution Product

According to the U.S. Surgeon General (2010 Report), the major smoking-related diseases, including cancer, heart and lung disease, have been linked to inhaling the combustion products of burning tobacco, rather than to nicotine per se. Therefore, less toxic forms of nicotine delivery, such as e-cigarettes, offer a potential avenue for harm reduction for smokers who cannot or do not wish to relinquish their nicotine dependence. A substantial fraction of smokers who use e-cigarettes continue to smoke combustible cigarettes (“dual use”), eroding the potential beneficial impact on disease risk. Therefore, it is important to examine strategies for enhancing complete switching to e-cigarettes from combustible cigarettes. It is believed that dual users find it most difficult to relinquish their favorite cigarettes, which surveys indicate are those smoked after meals. Jarvik M, et al. J Behav Med. 1993; 16(4): 413-422.

As shown in the clinical study herein, the combined treatment of a smoker with bupropion and zonisamide can facilitate the smoker to switch from smoking combustible cigarettes to a nicotine replacement or substitution product (e.g., e-cigarettes) and can help the smoker achieve and maintain abstinence from smoking combustible cigarettes.

In some embodiments, the present disclosure provides a method of treatment for facilitating a smoker to switch from combustible cigarettes to e-cigarettes and/or another nicotine replacement or substitution product, the method comprising administering to the smoker: a) bupropion or hydroxybupropion; and b) an anticonvulsant or a GABAergic agent. In some embodiments, the methods are for facilitating the smoker to switch from combustible cigarettes to e-cigarettes. In some embodiments, the methods are for facilitating the smoker to switch from combustible cigarettes to a nicotine replacement or substitution product, e.g., any of those described herein. For example, in some embodiments, the methods are for facilitating the smoker to switch from combustible cigarettes to one or more nicotine replacement or substitution products selected from an e-cigarette, nicotine patch, nicotine gum, nicotine lozenge, nicotine pouch, nasal spray nicotine, dissolvable nicotine product, sublingual nicotine tablet, nicotine inhaler (including nicotine salt inhaler, such as Philip Morris International's Platform 3 product), and a modified risk tobacco product, e.g., a smokeless tobacco (such as a chewable tobacco) or a noncombustible tobacco product such as a heat-not-burn system (e.g., Philip Morris International's IQOS). In such methods, the smoker is typically not restricted in the use of the one or more nicotine replacement or substitution products.

Smokers suitable to be treated with the methods herein are not particularly limited. Typically, the smoker wants to quit smoking combustible cigarettes. In some embodiments, the smoker uses both e-cigarettes and combustible cigarettes prior to the treatment. In some embodiments, the smoker smokes at least 10 commercially available cigarettes (combustible cigarettes) or equivalent per day for at least 12 months prior to the treatment. In some embodiments, the smoker has an expired air CO reading of at least 10 ppm prior to the treatment. In some embodiments, the smoker is not in need of treatment of obesity.

The methods herein can also be typically characterized by certain treatment effects. For example, in some embodiments, the methods herein can reduce the smoker's daily consumption of combustible cigarettes, for example, compared to the daily consumption prior to the treatment, the number of combustible cigarettes is reduced by 20% or more, or 50% or more, and up to 100%. In some embodiments, the smoker achieves abstinence from combustible cigarettes. In some embodiments, the methods herein can reduce the smoker's expired air CO level by 20% or more (e.g., about 50%) compared to baseline (i.e., the expired air CO level prior to the treatment). For example, in some embodiments, the methods can reduce the smoker's expired air CO level to be less than 5 ppm. In some embodiments, the methods herein can also reduce the smoker's urinary NNAL (4-(methylnitrosamino)-1-(3-pyridyl)-1-butanol) level by 20% or more (e.g., about 50%) compared to baseline. In some embodiments, the methods herein can reduce the smoker's craving for combustible cigarettes. For example, in some embodiments, the treated smoker can achieve an average per-item craving score less than or equal to 4 on the 7 point Shiffman-Jarvik Withdrawal Scale (or less than 50 on a 100-point Visual Analog Scale). In some embodiments, the average per-item craving score of the treated smoker is not increased by more than 1 point based on the 7 point Shiffman-Jarvik Withdrawal Scale (or by more than 10 points on a 100-point Visual Analog Scale). In any of the embodiments described herein, unless otherwise specified or contrary from context, the treatment effects described herein can be achieved and/or maintained during the treatment period and beyond, such as after 1, 2, 3, 4, 5, or 6 months of treatment. In any of the embodiments described herein, unless otherwise specified or contrary from context, the methods herein can also be characterized by reduced adverse effects associated with bupropion or hydroxybupropion. For example, in some embodiments, the methods herein can be characterized by reduced incidences of insomnia and/or agitation compared to treatment with bupropion alone. In some embodiments, the methods herein can also be characterized by reduced incidences of seizure compared to treatment with bupropion alone. The treatment effects described herein can be typically achieved through using an effective dosing regimen of bupropion or hydroxybupropion and the anticonvulsant or a GABAergic agent, including dosing amounts, types of formulations, etc., which include any of those described herein.

Typically, the methods for facilitating the switch comprise administering a pharmaceutical composition comprising an effective amount of bupropion to the smoker. In some embodiments, the method can include administering a pharmaceutical composition comprising an effective amount of the active metabolite of bupropion, hydroxybupropion (6-hydroxybupropion), to the smoker or a pharmaceutical composition comprising an effective amount of one or more prodrugs of hydroxybupropion. The bupropion useful for the methods herein is not particularly limited to any specific form. For example, bupropion in its free form or a pharmaceutically acceptable salt, such as an HCl salt, can be used in the methods herein. Bupropion typically exists as a racemic mixture, such as in the marketed product under the Brandname of Wellbutrin® or Zyban®. Suitable formulations of bupropion for the methods for facilitating the switch include any of those described herein.

The anticonvulsant or GABAergic agent that can be administered to the smoker for the methods for facilitating the switch is also not particularly limited. However, in preferred embodiments, the anticonvulsant or GABAergic agent is zonisamide. Other anticonvulsants or GABAergic agents that act similarly to zonisamide may also be used for the methods. As with bupropion, the zonisamide useful for the methods herein is also not particularly limited to a specific form. For example, zonisamide in its free form or a pharmaceutically acceptable salt, such as a sodium salt, can be used in the methods herein. Suitable formulations of zonisamide for the methods for promoting smoking cessation include any of those described herein.

### Bupropion and Zonisamide Dosing Regimen

The combined treatment with bupropion and zonisamide for the methods herein is not limited to any particular dosing regimen. The dosing regimen can be typically adjusted as needed to achieve one or more desired treatment effects as described herein.

Typically, the daily dose of bupropion for the methods herein (e.g., any of those described herein, such as those shown in [1]-[148] of the Brief Summary section, as applicable) ranges from about 100 mg to about 450 mg, such as about 150 mg, about 300 mg, about 450 mg, or any ranges between the recited values, e.g., about 150 mg to about 450 mg or about 300 mg to about 450 mg. The daily dose of bupropion is generally administered to the subject orally. The bupropion is typically administered as its pharmaceutically acceptable salt, such as hydrochloride salt (HCl salt) or hydrobromide salt (HBr salt). The bupropion may be administered in an immediate release formulation or a modified release formulation, such as a sustained release formulation or extended release formulation.

Bupropion formulations suitable for the methods herein include any of those known in the art, which include any of the formulations approved by the U.S. Food and Drug Administration (FDA) or a non-US counterpart agency, such as extended release APLENZIN® tablets (174 mg. 348 mg, or 522 mg of bupropion hydrobromide), ZYBAN® (bupropion hydrochloride) sustained-release tablets, FORFIVO® XL tablets (450 mg of bupropion hydrochloride), WELLBUTRIN® tablets (75 mg or 100 mg of bupropion hydrochloride), WELLBUTRIN® SR sustained-release tablets (100 mg, 150 mg, or 200 mg of bupropion hydrochloride), WELLBUTRIN® XL extended-release tablets (150 mg or 300 mg bupropion hydrochloride), or a generic bioequivalent product approved by the FDA for any of the foregoing. Non-limiting bupropion formulations suitable for the methods herein also include those bupropion formulations described in U.S. Pat. Nos. 5,427,798, 6,096,341, 6,143,327, 6,905,708, 7,579,380, and 8,932,628, the content of each of which is herein incorporated by reference in its entirety.

In some embodiments, the bupropion is administered in a sustained release formulation. Typically, a “sustained release” bupropion formulation herein includes dosage forms characterized in that a single administration can release bupropion in a way to result in plasma concentrations of bupropion and/or bupropion metabolite(s) maintained at a therapeutic level for a period of time such that the sustained release dosage form provides a therapeutic benefit (e.g., smoking cessation) over a period of about 6 hours or more, preferably, about 12 hours or more. Typically, the sustained release bupropion formulation herein is formulated for a twice-daily dosing regimen. In some embodiments, the sustained release bupropion formulation can be a tablet which includes bupropion hydrochloride and a release control polymer such as hydroxypropyl methylcellulose or hydroxypropyl cellulose, such as those described in U.S. Pat. No. 5,427,798, the content of which is herein incorporated by reference in its entirety. In some embodiments, the sustained release bupropion formulation can be a tablet which includes about 150 mg of bupropion hydrochloride and has the following inactive ingredients: Carnauba wax, cysteine hydrochloride, hydroxypropyl-methylcellulose, magnesium stearate, microcrystalline cellulose, polyethylene glycol, polysorbate 80, titanium dioxide, with a pharmacokinetic profile suitable for twice daily dosing. In some embodiments, the sustained release bupropion formulation can be a ZYBAN® (bupropion hydrochloride) sustained-release tablet or a generic bioequivalent product thereof.

In some embodiments, the bupropion is administered in an extended release formulation. Typically, an “extended release” bupropion formulation herein includes dosage forms characterized in that a single administration can release bupropion in a way to result in plasma concentrations of bupropion and/or bupropion metabolite(s) maintained at a therapeutic level for a period of time such that the extended release dosage form provides a therapeutic benefit (e.g., smoking cessation) over a period of about 12 hours or more, preferably, about 24 hours or more. Typically, the extended release bupropion formulation herein is formulated for a once-daily dosing regimen. In some embodiments, the extended release bupropion formulation can be a tablet which includes bupropion hydrochloride in a core, a film coating comprising ethyl cellulose, and a second coating comprising methacrylic acid co-polymer, such as those described in U.S. Pat. No. 6,143,327, the content of which is herein incorporated by reference in its entirety. In some embodiments, the extended release bupropion formulation can be a tablet which includes about 150 mg or 300 mg of bupropion hydrochloride and has the following inactive ingredients: ethylcellulose, glyceryl behenate, methacrylic acid copolymer dispersion, polyvinyl alcohol, polyethylene glycol, povidone, silicon dioxide, and triethyl citrate, with a pharmacokinetic profile suitable for once daily dosing. In some embodiments, the extended release bupropion formulation can be a WELLBUTRIN® XL extended-release tablet or a generic bioequivalent product thereof.

As would be understood by those skilled in the art, the daily dose of bupropion and dosing regimen for the methods herein (e.g., any of those described herein, such as those shown in [1]-[148] of the Brief Summary section, as applicable) can be adjusted together with that of zonisamide for efficacy and safety profile and convenience.

For example, in some preferred embodiments, the bupropion is administered once daily at a daily dose of about 300 mg to about 450 mg, such as about 300 mg, using an extended release formulation with a pharmacokinetic/pharmacodynamic profile suitable for such once daily dosing, such as the WELLBUTRIN® XL extended-release tablets or generic bioequivalent products thereof. In some embodiments, the bupropion can be administered once daily in the morning. In some embodiments, the bupropion can also be administered once daily in the evening, such as a few hours (e.g., 4 or 5 hours) before sleep. In some embodiments, the bupropion dosing may be combined with a concurrent or substantially simultaneous dosing of the anticonvulsant or GABAergic agent, preferably, zonisamide. In some embodiments, the bupropion dosing can also be administered to the subject before or after the administration of the anticonvulsant or GABAergic agent. Typically, the dose of the anticonvulsant or GABAergic agent, preferably, zonisamide, provides a suitable pharmacokinetic profile such that the side effect associated with the administration of the bupropion extended release formulation, such as insomnia, can be reduced, minimized, or eliminated.

In some embodiments, the bupropion can also be administered twice daily at a daily dose of about 300 mg to about 450 mg, such as about 300 mg, using a sustained release formulation with a pharmacokinetic/pharmacodynamic profile suitable for such twice daily dosing, such as the ZYBAN® (bupropion hydrochloride) sustained-release tablets or generic bioequivalent products thereof. In such embodiments, the daily dose of bupropion is typically administered in two equivalent doses, with one in the morning and one in the evening, such as several hours (e.g., 8 hours) after the first dose. In some embodiments, one or both dosing occasions may be combined with a concurrent or substantially simultaneous dosing of the anticonvulsant or GABAergic agent, preferably, zonisamide. In some embodiments, one or both dosing can also be administered to the subject before or after the administration of the anticonvulsant or GABAergic agent. Typically, the dose of the anticonvulsant or GABAergic agent, preferably, zonisamide, provides a suitable pharmacokinetic profile such that the side effect associated with the administration of the bupropion sustained release formulation, such as insomnia, can be reduced, minimized, or eliminated.

In some embodiments, the bupropion can also be administered as an immediate release formulation, for example, to achieve an acute prophylaxis against smoking relapse in stressful situations or other craving-eliciting situations. In some embodiments, the bupropion immediate release formulation can be administered to the subject in the evening, such as a few hours (e.g., 4 or 5 hours) before sleep. In some embodiments, the bupropion immediate release formulation can also be combined with a concurrent or substantially simultaneous dosing of the anticonvulsant or GABAergic agent, preferably, zonisamide. In some embodiments, the bupropion immediate release formulation can also be administered to the subject before or after the administration of the anticonvulsant or GABAergic agent, preferably, zonisamide. Typically, the dose of the anticonvulsant or GABAergic agent, preferably, zonisamide, provides a suitable pharmacokinetic profile such that the side effect associated with the administration of the bupropion immediate release formulation, such as insomnia, can be reduced, minimized, or eliminated.

The daily dose of zonisamide for the methods herein (e.g., any of those described herein, such as those shown in [1]-[148] of the Brief Summary section, as applicable) generally ranges from about 25 mg to about 400 mg. e.g., about 25 mg, about 50 mg, about 75 mg, about 100 mg, about 120 mg, about 150 mg, about 200 mg, about 250 mg, about 300 mg, about 400 mg, or any ranges between the recited values, e.g., about 25 mg to about 100 mg, about 50 mg to about 100 mg, etc. The daily dose of zonisamide is typically administered to the subject orally. Zonisamide formulations suitable for the methods herein include any of those known in the art, including any of the formulations approved by the U.S. Food and Drug Administration (FDA) or a non-US counterpart agency, such as immediate release ZONEGRAN® (zonisamide) capsules containing 25 mg, 50 mg, or 100 mg zonisamide, or a generic bioequivalent product approved by the FDA. In some embodiments, the zonisamide can be present in a formulation (such as capsules) containing 25 mg. 50 mg or 100 mg zonisamide and has the following inactive ingredients: microcrystalline cellulose, hydrogenated vegetable oil, sodium lauryl sulfate, gelatin, and colorants. Although typically zonisamide as used herein is presented in an immediately release formulation, in some embodiments, the zonisamide can also be present in a sustained release formulation. Suitable sustained release formulation includes those known in the art, such as those described in United States Published Patent Application No. US 2007/0148237 A1, the content of which is herein incorporated by reference in its entirety. Typically, the zonisamide is administered once daily. In some embodiments, the zonisamide can be administered once daily in the morning. In some embodiments, the zonisamide can also be administered once daily in the evening, such as a few hours (e.g., 4 or 5 hours) before sleep.

For the methods herein (e.g., any of those described herein, such as those shown in [1]-[148] of the Brief Summary section, as applicable), the zonisamide can be administered to the subject concurrently with bupropion or sequentially in any order. For example, in some embodiments, a dosage form comprising both zonisamide and bupropion as active ingredients can be administered to the subject. Dosage forms containing both zonisamide and bupropion as active ingredients are known, such as those layered tablets described in U.S. Pat. Nos. 8,318,788 B2 and 8,088,786 B2. In some embodiments, separate dosage forms comprising zonisamide and bupropion, respectively, can be administered to the subject concurrently. In some embodiments, zonisamide can be administered to the subject before or after bupropion.

Typically, for sequential administration of zonisamide and bupropion according to the methods herein, the zonisamide and bupropion can be administered to the subject about a few hours apart within the same day, such as about 1, 2, 4, 6, 8, 10, or 12 hours apart. However, in some embodiments, it is also contemplated that bupropion and zonisamide are not administered during the same day. For example, in some embodiments, the subject may be treated with bupropion once a day for a first period of time without zonisamide, which is then followed by a second period of time wherein the subject is treated with zonisamide once a day with or without bupropion. Similarly, in some embodiments, the subject may also be treated with zonisamide once a day for a first period of time without bupropion, which is then followed by a second period of time wherein the subject is treated with bupropion once a day with or without zonisamide. In some embodiments, there can also be a gap period between the first and second periods of time. In some embodiments, there is no gap period between first and second periods of time.

The weight ratio of the daily dose of zonisamide to bupropion for the methods herein (e.g., any of those described herein, such as those shown in [1]-[148] of the Brief Summary section, as applicable) can typically range from about 20:1 to about 1:20, preferably, from about 1:1 to about 1:10, such as about 1:1, about 1:2, about 1:3, about 1:4, about 1:5, about 1:6, about 1:7, about 1:8, about 1:9, about 1:10, or any ranges between the recited ratios, with bupropion being the higher daily dose. For example, in some embodiments, the daily dose of zonisamide can be about 50 mg and the daily dose of bupropion can be about 300 mg, i.e., a ratio of about 1:6. In some embodiments, the daily dose of zonisamide can be about 100 mg and the daily dose of bupropion can be about 300 mg. i.e., a ratio of about 1:3.

One advantage of the methods herein is the reduced incidences of adverse events associated with either bupropion or zonisamide. This can be achieved by adjusting the zonisamide and bupropion dosing amount, timing, formulation etc. such that the side effects of the two drugs offset each other. For example, in some embodiments according to the methods herein (e.g., any of those described herein, such as those shown in [1]-[148] of the Brief Summary section, as applicable), the zonisamide is administered in an amount effective in reducing one or more adverse effects associated with bupropion or hydroxybupropion, such as insomnia. In some embodiments, the zonisamide is administered in an amount effective in reducing incidences of insomnia and/or agitation without increasing incidences of somnolence or fatigue. In some embodiments, the zonisamide is administered in an amount effective in reducing incidences of seizure.

In some embodiments, the zonisamide can also be administered in an amount effective in enhancing smoking cessation effects of bupropion.

In typical embodiments according to the methods herein, both zonisamide and bupropion are administered to the subject orally. However, in some embodiments, one or both zonisamide and bupropion can also be administered to the subject through other routes of administration, such as sublingually, rectally, parentally (including subcutaneously, intramuscularly and intravenously), or transdermally.

In some specific embodiments, the present disclosure provides a method of treatment for promoting smoking cessation in a subject, the method comprises administering to the subject a) a daily dose of bupropion of about 150 mg to about 450 mg (e.g., about 300 mg to about 450 mg) in an extended release formulation (e.g., described herein) once daily, b) a daily dose of zonisamide of about 25 mg to about 400 mg (e.g., about 25 mg to about 100 mg or about 50 mg to about 100 mg) in an immediate release formulation (e.g., described herein) once daily, and c) optionally a nicotine replacement or substitution product (e.g., described herein). In some embodiments, the weight ratio of the daily dose of zonisamide to bupropion ranges from about 1:1 to about 1:10, such as about 1:3 to about 1:6. The zonisamide and bupropion are typically administered to the subject orally. In some embodiments, the zonisamide and bupropion are administered to the subject concurrently. In some embodiments, the zonisamide is administered before or after the bupropion. In some embodiments, the bupropion is administered in the morning. In some embodiments, the bupropion is administered in the evening. In some embodiments, the zonisamide is administered in the morning. In some embodiments, the zonisamide is administered in the evening. In some embodiments, the method comprises administering to the subject the nicotine replacement or substitution product, such as an e-cigarette, nicotine patch, nicotine gum, nicotine lozenge, nicotine pouch, nasal spray nicotine, dissolvable nicotine product, sublingual nicotine tablet, nicotine inhaler (including nicotine salt inhaler, such as Philip Morris International's Platform 3 product), and/or a modified risk tobacco product (e.g., smokeless tobacco such as chewable tobacco or noncombustible tobacco product). In some embodiments, the method does not administer to the subject the nicotine replacement or substitution product. Characteristics of the subject and treatment effects suitable for the method include any of those described herein.

In some specific embodiments, the present disclosure provides a method of treatment for reducing a subject's craving for combustible cigarettes, the method comprises administering to the subject a) a daily dose of bupropion of about 150 mg to about 450 mg (e.g., about 300 mg to about 450 mg) in an extended release formulation (e.g., described herein) once daily, b) a daily dose of zonisamide of about 25 mg to about 400 mg (e.g., about 25 mg to about 100 mg or about 50 mg to about 100 mg) in an immediate release formulation (e.g., described herein) once daily, and c) optionally a nicotine replacement or substitution product (e.g., described herein). In some embodiments, the weight ratio of the daily dose of zonisamide to bupropion ranges from about 1:1 to about 1:10, such as about 1:3 to about 1:6. The zonisamide and bupropion are typically administered to the subject orally. In some embodiments, the zonisamide and bupropion are administered to the subject concurrently. In some embodiments, the zonisamide is administered before or after the bupropion. In some embodiments, the bupropion is administered in the morning. In some embodiments, the bupropion is administered in the evening. In some embodiments, the zonisamide is administered in the morning. In some embodiments, the zonisamide is administered in the evening. In some embodiments, the method comprises administering to the subject the nicotine replacement or substitution product, such as an e-cigarette, nicotine patch, nicotine gum, nicotine lozenge, nicotine pouch, nasal spray nicotine, dissolvable nicotine product, sublingual nicotine tablet, nicotine inhaler (including nicotine salt inhaler, such as Philip Morris International's Platform 3 product), and/or a modified risk tobacco product (e.g., smokeless tobacco such as chewable tobacco or noncombustible tobacco product). In some embodiments, the method does not administer to the subject the nicotine replacement or substitution product. Characteristics of the subject and treatment effects suitable for the method include any of those described herein.

In some specific embodiments, the present disclosure provides a method of treating dependency, addiction, or withdrawal associated with combustible cigarettes in a subject, the method comprises administering to the subject a) a daily dose of bupropion of about 150 mg to about 450 mg (e.g., about 300 mg to about 450 mg) in an extended release formulation (e.g., described herein) once daily, b) a daily dose of zonisamide of about 25 mg to about 400 mg (e.g., about 25 mg to about 100 mg or about 50 mg to about 100 mg) in an immediate release formulation (e.g., described herein) once daily, and c) optionally a nicotine replacement or substitution product (e.g., described herein). In some embodiments, the weight ratio of the daily dose of zonisamide to bupropion ranges from about 1:1 to about 1:10, such as about 1:3 to about 1:6. The zonisamide and bupropion are typically administered to the subject orally. In some embodiments, the zonisamide and bupropion are administered to the subject concurrently. In some embodiments, the zonisamide is administered before or after the bupropion. In some embodiments, the bupropion is administered in the morning. In some embodiments, the bupropion is administered in the evening. In some embodiments, the zonisamide is administered in the morning. In some embodiments, the zonisamide is administered in the evening. In some embodiments, the method comprises administering to the subject the nicotine replacement or substitution product, such as an e-cigarette, nicotine patch, nicotine gum, nicotine lozenge, nicotine pouch, nasal spray nicotine, dissolvable nicotine product, sublingual nicotine tablet, nicotine inhaler (including nicotine salt inhaler, such as Philip Morris International's Platform 3 product), and/or a modified risk tobacco product (e.g., smokeless tobacco such as chewable tobacco or noncombustible tobacco product). In some embodiments, the method does not administer to the subject the nicotine replacement or substitution product. Characteristics of the subject and treatment effects suitable for the method include any of those described herein.

In some specific embodiments, the present disclosure provides a method of reducing the rewarding effects of smoking (e.g., satisfaction, enjoyment of the taste, enhanced attention, reduced irritability) and/or increasing the aversive effects of smoking (e.g. nausea, dizziness) of combustible cigarettes in a subject, the method comprises administering to the subject a) a daily dose of bupropion of about 150 mg to about 450 mg (e.g., about 300 mg to about 450 mg) in an extended release formulation (e.g., described herein) once daily, b) a daily dose of zonisamide of about 25 mg to about 400 mg (e.g., about 25 mg to about 100 mg or about 50 mg to about 100 mg) in an immediate release formulation (e.g., described herein) once daily, and c) optionally a nicotine replacement or substitution product (e.g., described herein). In some embodiments, the weight ratio of the daily dose of zonisamide to bupropion ranges from about 1:1 to about 1:10, such as about 1:3 to about 1:6. The zonisamide and bupropion are typically administered to the subject orally. In some embodiments, the zonisamide and bupropion are administered to the subject concurrently. In some embodiments, the zonisamide is administered before or after the bupropion. In some embodiments, the bupropion is administered in the morning. In some embodiments, the bupropion is administered in the evening. In some embodiments, the zonisamide is administered in the morning. In some embodiments, the zonisamide is administered in the evening. In some embodiments, the method comprises administering to the subject the nicotine replacement or substitution product, such as an e-cigarette, nicotine patch, nicotine gum, nicotine lozenge, nicotine pouch, nasal spray nicotine, dissolvable nicotine product, sublingual nicotine tablet, nicotine inhaler (including nicotine salt inhaler, such as Philip Morris International's Platform 3 product), and/or a modified risk tobacco product (e.g., smokeless tobacco such as chewable tobacco or noncombustible tobacco product). In some embodiments, the method does not administer to the subject the nicotine replacement or substitution product. Characteristics of the subject and treatment effects suitable for the method include any of those described herein.

In some specific embodiments, the present disclosure provides a method of treatment for facilitating a smoker to switch from combustible cigarettes to e-cigarettes, the method comprises administering to the smoker a) a daily dose of bupropion of about 150 mg to about 450 mg (e.g., about 300 mg to about 450 mg) in an extended release formulation (e.g., described herein) once daily, and b) a daily dose of zonisamide of about 25 mg to about 400 mg (e.g., about 25 mg to about 100 mg or about 50 mg to about 100 mg) in an immediate release formulation (e.g., described herein) once daily. In some embodiments, the weight ratio of the daily dose of zonisamide to bupropion ranges from about 1:1 to about 1:10, such as about 1:3 to about 1:6. The zonisamide and bupropion are typically administered to the smoker orally. In some embodiments, the zonisamide and bupropion are administered to the smoker concurrently. In some embodiments, the zonisamide is administered before or after the bupropion. In some embodiments, the bupropion is administered in the morning. In some embodiments, the bupropion is administered in the evening. In some embodiments, the zonisamide is administered in the morning. In some embodiments, the zonisamide is administered in the evening. Characteristics of the smoker and treatment effects suitable for the method include any of those described herein.

In some specific embodiments, the present disclosure provides a method of treatment for facilitating a smoker to switch from combustible cigarettes to one or more nicotine replacement or substitution products selected from an e-cigarette, nicotine patch, nicotine gum, nicotine lozenge, nicotine pouch, nasal spray nicotine, dissolvable nicotine product, sublingual nicotine tablet, nicotine inhaler (including nicotine salt inhaler, such as Philip Morris International's Platform 3 product), and a modified risk tobacco product, e.g., a smokeless tobacco such as chewable tobacco or a noncombustible tobacco product such as a heat-not-burn system (e.g., Philip Morris International's IQOS), the method comprises administering to the smoker a) a daily dose of bupropion of about 150 mg to about 450 mg (e.g., about 300 mg to about 450 mg) in an extended release formulation (e.g., described herein) once daily, and b) a daily dose of zonisamide of about 25 mg to about 400 mg (e.g., about 25 mg to about 100 mg or about 50 mg to about 100 mg) in an immediate release formulation (e.g., described herein) once daily. In some embodiments, the weight ratio of the daily dose of zonisamide to bupropion ranges from about 1:1 to about 1:10, such as about 1:3 to about 1:6. The zonisamide and bupropion are typically administered to the smoker orally. In some embodiments, the zonisamide and bupropion are administered to the smoker concurrently. In some embodiments, the zonisamide is administered before or after the bupropion. In some embodiments, the bupropion is administered in the morning. In some embodiments, the bupropion is administered in the evening. In some embodiments, the zonisamide is administered in the morning. In some embodiments, the zonisamide is administered in the evening. Characteristics of the smoker and treatment effects suitable for the method include any of those described herein.

### Nicotine Replacement or Substitution Product

Suitable nicotine replacement or substitution products for the methods herein (e.g., any of those described herein, such as those shown in [1]-[148] of the Brief Summary section, as applicable) are not particularly limited and broadly includes any products that can deliver nicotine to a subject user through non-combustible ways. For example, the nicotine replacement or substitution product can be an e-cigarette, nicotine patch, nicotine gum, nicotine lozenge, nicotine pouch, nasal spray nicotine, dissolvable nicotine product, sublingual nicotine tablet, and/or nicotine inhaler (including nicotine salt inhaler, such as Philip Morris International's Platform 3 product).

In some preferred embodiments according to the methods herein (e.g., any of those described herein, such as those shown in [1]-[148] of the Brief Summary section, as applicable), the nicotine replacement or substitution product is an e-cigarette. Suitable e-cigarettes are not particularly limited and include any of those known in the art, such as Halo G6 electronic nicotine delivery system, e.g., described herein, or any of the commercially available e-cigarettes. E-cigarettes typically include an atomizer, a power source such as a battery, and a container for the e-liquid such as a cartridge or tank. The e-liquid typically includes nicotine, propylene glycol, glycerin, and flavors. See e.g., U.S. Pat. Nos. 10,952,468, 10,463,069, etc.

In some embodiments, the nicotine replacement or substitution product can also be a nicotine replacement therapy, which includes for example, (1) nicotine transdermal patches, such as NicoDerm® CQ® (GlaxoSmithKline), Habitrol® (Novartis Consumer Health), and Nicotrol® (Pharmacia Consumer Healthcare); (2) nicotine gum, such as Nicorette® (GlaxoSmithKline); (3) nicotine nasal spray, such as Nicotrol NS® (Pharmacia Consumer Healthcare); and (4) nicotine inhaler (Nicotrol® nicotine inhalation system (Pharmacia Consumer Healthcare).

In some embodiments, the nicotine replacement or substitution product can also be a modified risk tobacco product. For example, in some embodiments, the modified risk tobacco product is a smokeless tobacco such as chewable tobacco or a noncombustible tobacco product such as a heat-not-burn system (e.g., Philip Morris International's IQOS).

In some embodiments, the nicotine replacement or substitution product can also be a nicotine salt delivery system, for example, a nicotine-based electronic-free inhaler product, which mechanically produces a nicotine-containing aerosol without tobacco, combustion, or heating such as Philip Morris International's platform 3 product. Such nicotine-based electronic-free product, such as Platform 3, can typically be composed of two parts; a consumable that contains highly-soluble encapsulated nicotine powder, and a non-electronic device that activates it.

When a nicotine replacement or substitution product is used in the methods herein, the amount, frequency, and type of such product is not particularly limited. For example, when the nicotine replacement or substitution product is an e-cigarette, the use of the e-cigarette is typically ad libitum. In some cases, two or more of such replacement or substitution products can be used.

### Kits

In some embodiments, the present disclosure also provides kits useful for the methods described herein. For example, in some embodiments, the present disclosure provides a kit comprising: a) bupropion; b) zonisamide; and optionally c) a nicotine replacement or substitution product. The bupropion included in the kit can be present in any of the bupropion formulations described herein, such as a sustained release or extended release bupropion formulation. Similarly, the zonisamide and nicotine replacement or substitution product can also be any of those described herein, respectively. In some embodiments, the kit can include a) one or more daily doses of bupropion, each comprising an extended release formulation (e.g., described herein) comprising about 150 mg to about 450 mg bupropion; b) one or more daily doses of zonisamide, each comprising a pharmaceutical composition comprising about 25 mg to about 400 mg zonisamide, and optionally c) a nicotine replacement or substitution product. In some embodiments, the kit can include a) one or more daily doses of bupropion, each comprising an extended release formulation (e.g., described herein) comprising about 300 mg to about 450 mg bupropion, suitable for once daily dosing; b) one or more daily doses of zonisamide, each comprising a pharmaceutical composition comprising about 25 mg to about 400 mg zonisamide, such as about 25 mg to about 100 mg or about 50 mg to about 100 mg of zonisamide; and optionally c) a nicotine replacement or substitution product. In some embodiments, the kit can include a) one or more daily doses of bupropion, each comprising an extended release formulation comprising about 300 mg to about 450 mg bupropion, suitable for once daily dosing; and b) one or more daily doses of zonisamide, each comprising a pharmaceutical composition comprising about 25 mg to about 400 mg zonisamide, such as about 25 mg to about 100 mg or about 50 mg to about 100 mg of zonisamide, and does not include a nicotine replacement or substitution product. In some embodiments, the kit can further include an instruction on how to use the kit, for example, an instruction stating that the kit is for promoting smoking cessation, reducing a subject's craving for combustible tobacco products, such as cigarettes, for treating dependency, addiction, or withdrawal associated with combustible tobacco products, such as cigarettes, for reducing rewarding effects of smoking (e.g., satisfaction, enjoyment of the taste, enhanced attention, reduced irritability) and/or increasing the aversive effects of smoking (e.g. nausea, dizziness) of combustible cigarettes, or for facilitating a smoker to switch from combustible cigarettes to e-cigarettes or another nicotine replacement or substitution product, etc.

### Definitions

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value.

The term “subject” or “smoker” is used interchangeably herein and should be understood as referring to a human.

### EXAMPLES

### Example 1. Zonisamide/Bupropion Effects on Switching to Electronic Cigarettes

This example shows an open-label study exploring the impact of combination zonisamide and bupropion on the process of switching from combustible cigarettes (CC) to an e-cigarette, using the protocol below.

### Clinical Protocol

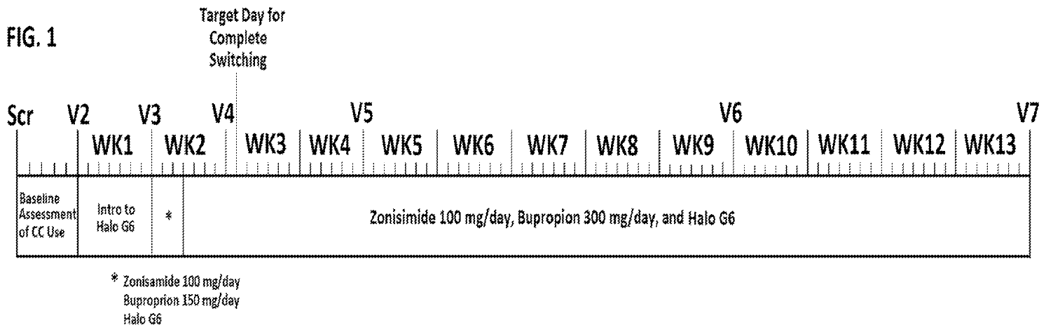

Overall Study Design This single-group, small-scale, open-label study (N=24) evaluated the impact of combination zonisamide and bupropion on the process of switching from combustible cigarettes (CC) to an e-cigarette. There was a data collection period of at least five days to obtain baseline data on use of combustible cigarettes. Participants enrolled in the study received a G6 e-cigarette at V2 for ad libitum use. After the first week of e-cigarette use, (at V3) participants were given zonisamide (100 mg/daily, manufactured by Glenmark Pharmaceuticals, 750 Corporate Drive, Mahwah, NJ 07430, distributed by Cardinal Health, 7000 Cardinal Place, Dublin, OH 43017) and began extended-release bupropion dosing (150 mg each morning days 1-3, then 300 mg/daily, manufactured by Lupin Pharmaceuticals, Inc.; US Headquarters: Harborplace Tower, 111 S. Calvert Street, 21st Floor, Baltimore, MD 21202, distributed by Cardinal Health, 7000 Cardinal Place, Dublin, OH 43017) in addition to continued use of the G6. At each visit, participants received enough zonisamide, bupropion, and Halo G6 cartomizers to last until their next study visit. Halo G6 and combination zonisamide and bupropion use continued until the participant returns for the End-of-Study visit (V7). The overall study design is also shown in FIG. 1.

At each session, expired air CO was measured along with blood pressure, heart rate, respiratory rate and body weight. Participants also completed questionnaires rating subjective effects of smoking and e-cigarette use. Participants were given enough cartomizers and study drugs to last until their next scheduled session, along with an extra 4 days' worth to allow for flexibility in case sessions need to be rescheduled. E-cigarette usage and study drug compliance were tracked not only by self-reported number of occasions used, but also by cartomizer counts (used or unused) and returned study drugs.

Inclusion and Exclusion Criteria Healthy, cigarette smoking adults, age 21-65 years, with no restriction on gender, race and ethnicities, or social-economic status, who had smoked an average of at least 10 commercially available cigarettes (combustible cigarettes) per day for the last 12 months were screened for enrollment in this study.

Each participant must meet all the following inclusion criteria before enrollment:

Inclusion Criteria

| | |
|---|---|
| 1. | Has signed the ICF and is able to read and understand the |
| | information provided in the ICF. |
| 2. | Is 21 to 65 years of age (inclusive) at screening. |
| 3. | Smokes at least 10 commercially available cigarettes per day (no |
| | brand restrictions), for the last 12 months. |
| 4. | Has an expired air CO reading of at least 10 ppm at screening. |
| 5. | Interested in switching to an electronic cigarette. |
| 6. | Willing and able to comply with the requirements of the study. |
| 7. | Owns a smart phone with text message and data capabilities |
| | compatible with necessary surveys. |

Exclusion criteria include the following:

Exclusion Criteria

| | |
|---|---|
| 1. | Is unhealthy or cannot participate in the study for any reason (e.g., |
| | medical, psychiatric, and/or social reason) as judged by the |
| | Investigator or designated medical staff based on all available |
| | assessments from the screening period (e.g., safety laboratory, |
| | vital signs, physical examination, ECG, concomitant medications |
| | and medical history). |
| 2. | PHQ-9 score greater than 9, or a score greater than 0 on item #9 |
| | (“Thoughts that you would be better off dead, or of hurting |
| | yourself in some way”) at screening. |
| 3. | Planned use of an FDA-approved smoking cessation product during |
| | the study. |
| 4. | High blood pressure (systolic >150 mmHg or diastolic >95 mmHg) |
| | at screening. |
| 5. | Body mass index (BMI) less than 15.0 kg/m² or greater than |
| | 40.0 kg/m². |
| 6. | Coronary heart disease, structural cardiac disease (including, |
| | but not limited to valvular heart disease or cardiac murmurs), |
| | cardiac dysrhythmias, syncope, cardiac chest pain, or history of |
| | heart attack or heart failure. |
| 7. | Has received psychotherapy or behavioral treatments potentially |
| | impacting symptoms of depression, anxiety, or nicotine withdrawal |
| | within 30 days of screening, or during the study. |
| 8. | Taking antidepressants, psychoactive medications (e.g. |
| | antipsychotics, benzodiazepines, hypnotics) or medications |
| | that prolong QT_{c}. |
| 9. | Use of any of these products in the past 30 days: |
| | a. Illegal drugs (or if the urine drug screen is positive for |
| | cocaine, THC, amphetamines, methamphetamines, or opiates); |
| | b. Experimental (investigational) drugs that are unknown to |
| | participant; |
| | c. Chronic opiate use. |
| 10. | Use of smokeless tobacco (chewing tobacco, snuff), cigars (except |
| | for “Black & Mild” cigars or Cigarillos), pipes, hookah, |
| | e-cigarettes, nicotine replacement therapy or other smoking cessation |
| | treatments within 14 days of screening. |
| 11. | Pregnant or nursing (by self-report) or has a positive pregnancy test. |
| 12. | Enrollment requirements met. |

HALO-G6 ELECTRONIC NICOTINE DELIVERY SYSTEM The Halo G6 is a breath-actuated, rechargeable e-cigarette that comes with prefilled e-liquid cartomizers. This e-cigarette was chosen over other tank-based or pod-based e-cigarette models because of its similarity in shape and size to a cigarette. Because one of the goals is to provide habit substitution for smoking that zonisamide/bupropion cannot provide, this “cigalike” design is considered advantageous. Each G6 prefilled cartomizer contains a 50/50 blend of propylene glycol and vegetable glycerin containing a nicotine salt with 35 mg/mL nicotine strength. The 3.5% nicotine concentration was chosen over higher (e.g. 5%) concentrations to reduce the likelihood of nausea. The cartomizers come in packs of five. All Halo brand e-liquids undergo independent testing and they are manufactured by Nicopure labs. This study used “Tribeca” (tobacco) and “Menthol” flavored cartomizers, with participants matched to their preferred cigarette flavor. At Visit 2, participants were allowed to use the device ad libitum for a maximum of 10 minutes. Research staff inquired whether the participants were willing and able to use the Halo G6 during the study, to determine eligibility. The maximum amount of time the G6 in use was for 13 weeks, plus up to an additional four days (to allow for the scheduling window). Participants were instructed on how to use the e-cigarette prior to dispensing.

ZONISAMIDE Zonisamide is currently marketed as an antiepileptic medication for treatment of partial seizures. Dosing of zonisamide remained unchanged for the duration of the 12-week dosing period unless changed per guidance below. Participants were expected to take 100 mg (two 50 mg capsules) orally once a day. Zonisamide was dispensed in the form of a 50 mg capsules. Adjustments could be made to the timing of the dose (AM or PM) or the amount (1 or 2 capsules) depending on the somnolence/activation experienced by each individual participant. Initially, both bupropion and zonisamide were to be taken in the morning. Should participants experience significant drowsiness, the medical providers (MD/PA) could change the dosing to nighttime (2 hours prior to bedtime) or decrease the dose to one 50 mg capsule per day. All dosing adjustments would be approved by the Medical Director for this study.

BUPROPION Bupropion inhibits reuptake of both noradrenaline and dopamine, and also produces some blockade of nicotinic acetylcholinergic receptors. This medication has been approved as an antidepressant for over 20 years. The Food and Drug Administration approved the use of bupropion for smoking cessation in 1997. The usual dose for bupropion for smoking cessation is 150 mg once a day for 3 days, followed by 300 mg once a day for 7 to 12 weeks. The maximum prescribed dose for bupropion is 450 mg a day, which is the maximum dose for treating major depressive disorder and attention deficit hyperactivity disorder for adults. The dosage for this study: 150 mg bupropion tablet orally once a day for 3 days; then two 150 mg tablets (300 mg) orally once a day for the remainder of study participation. Bupropion tablets were dispensed in the form of a 150 mg extended release tablet, initially one tablet daily for 3 days, followed by 2 tablets daily until End of Study. Extended release tablets have a pharmacokinetic profile that supports once daily dosing. Combining this medication with zonisamide would decrease the risk for side-effects, allowing for daily dosing, which would likely improve compliance. Adjustments may be made depending on the side-effects experienced by each individual participant. Initially, both bupropion and zonisamide were to be taken in the morning. Should participants experience significant activation during the day, the medical providers (MD/PA) could either adjust the dose down to 150 mg daily or split the dose (150 mg taken twice daily which is normal dosing for smoking cessation). All dosing adjustments would be approved by the Medical Director for this study.

### Assessments

An overview of all assessments is provided in the schedule of events:

| | | | | | | | | | | Product |
|---|---|---|---|---|---|---|---|---|---|---|
| | | Screening | Baseline | | | | | | | Use Period |
| | | Session | Cig Data | Laboratory Sessions | | | | | | 13 Weeks |
| Visit Assessments and Procedures | | V1 | Collection | V2 | V3 | V4 | V5 | V6 | V7 | PMSMS |
| Informed Consent and Guidance | | • | | | | | | | | |
| Inclusion/Exclusion Criteria | | • | | | | | | | | |
| Enrollment/Randomization | | | | • | | | | | | |
| Prior and Concomitant Medication | | • | | • | • | • | • | • | • | |
| Questionaires | Smoking History Questionnaire | • | | | | | | | | |
| | Registration Form | • | | | | | | | | |
| | Employment History | | | • | | | | | | |
| | Medical History/Review of Systems | • | | | | | | | | |
| | Payment Verification Form | • | | • | • | • | • | • | • | |
| | Reasons to Smoke | | | • | | | | | •* | |
| | Modified Cigarette Evaluation | | | • | • | • | • | • | • | |
| | Questionnaire Extended (mCEQE) | | | | | | | | | |
| | Modified e-Cigarette Evaluation | | | | • | • | • | • | • | |
| | Questionnaire Extended (meCEQE) | | | | | | | | | |
| | The Fagerström Test for | | | • | • | | | | • | |
| | Nicotine Dependence (FTND) | | | | | | | | | |
| | Shiffman-Jarvik Withdrawal Scale | | | • | • | • | • | • | • | |
| | Assessment of Behavioral OUTcomes (ABOUT) | | | • | • | | | | • | |
| | Patient Health Questionnaire (PHQ-9) | • | | • | • | • | • | • | • | |
| | e-Cigarette Usage and Smoking | | • | | | | | | | • |
| | Status (via daily SMS text) | | | | | | | | | |
| | Medication Adherence (via daily SMS text) | | | | | | | | | • |
| Safety Laboratories | | • | | | | | | | | |
| Serum Pregnancy Test (Females) | | • | | | | | | | | |
| Urine Pregnancy Test (Females) | | | | • | • | • | • | • | • | |
| Urine Drug Screen | | • | | | | | | | | |
| CO Breath Test | | • | | • | • | • | • | • | • | |
| ECG | | • | | | | | | | | |
| Vitals | Blood Pressure | • | | • | • | • | • | • | • | |
| | Heart rate | • | | • | • | • | • | • | • | |
| | Temperature | • | | | | | | | | |
| | Respiratory rate | • | | • | • | • | • | • | • | |
| | Weight | • | | • | • | • | • | • | • | |

| | | | | | | | |
|---|---|---|---|---|---|---|---|
| Height | • | | | | | | |
| Physical Examination | • | •^{#} | •^{#} | •^{#} | •^{#} | •^{#} | •^{#} |
| Halo Assessment and Questionnaire | | • | | | | | |
| Collect Used/Unused Halo products | | | • | • | • | • | • |
| Dispense Halo products | | • | • | • | • | • | |
| Collect Used/Unused Blister Packs | | | | • | • | • | • |
| Dispense Study Drugs in Blister Packs | | | • | • | • | • | |

Expired Air CO Breath Test Carbon Monoxide (CO) in participant's exhaled breath (expressed as ppm) were measured using a Vitalograph CO Monitor. Participants must have an expired air CO reading at V1 of at least 10 ppm for inclusion into this study. This test were repeated at each of the sessions.

AE/SAE Reporting AEs/SAEs were assessed using questionnaires and interviews at the indicated time points and spontaneous reporting from the time of ICF signature until the EOS for the participant.

The questionnaires were administered to the participants using paper questionnaires and/or an electronic data collection system.

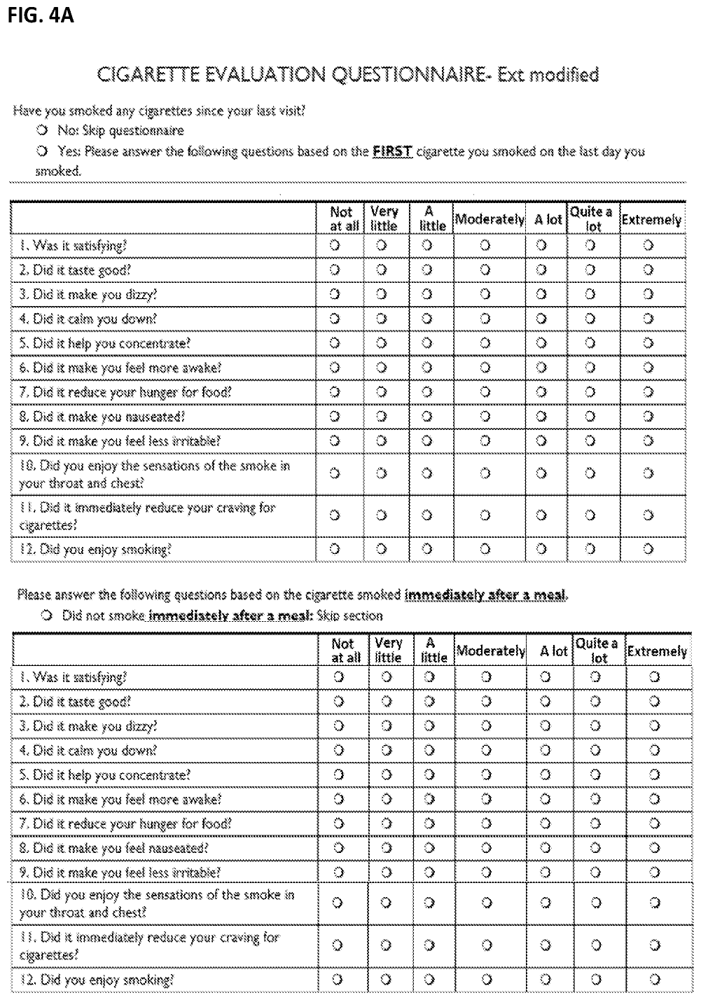

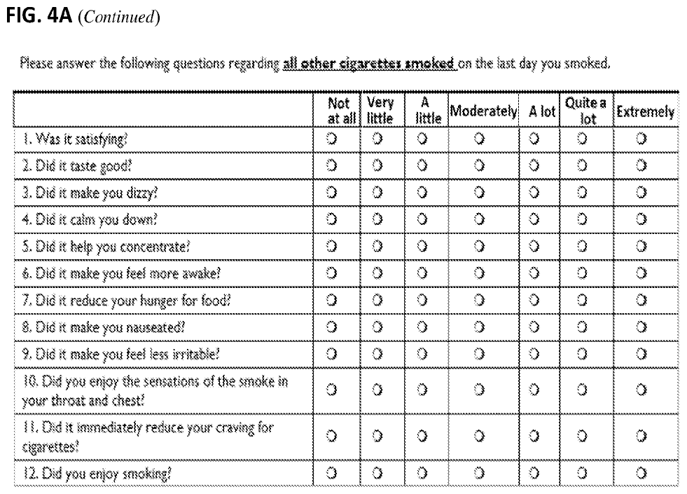

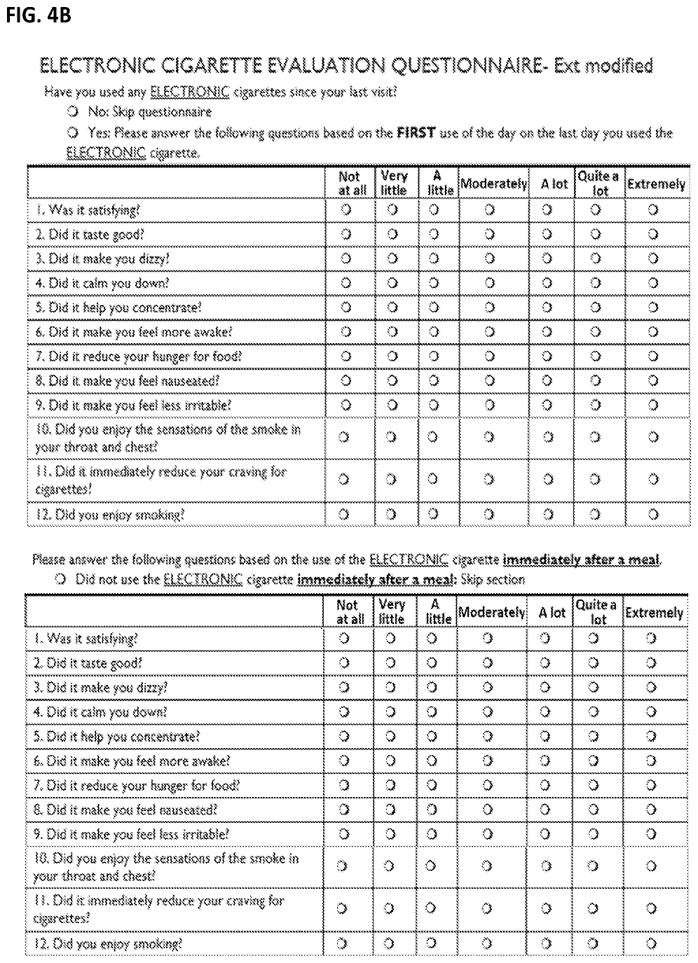

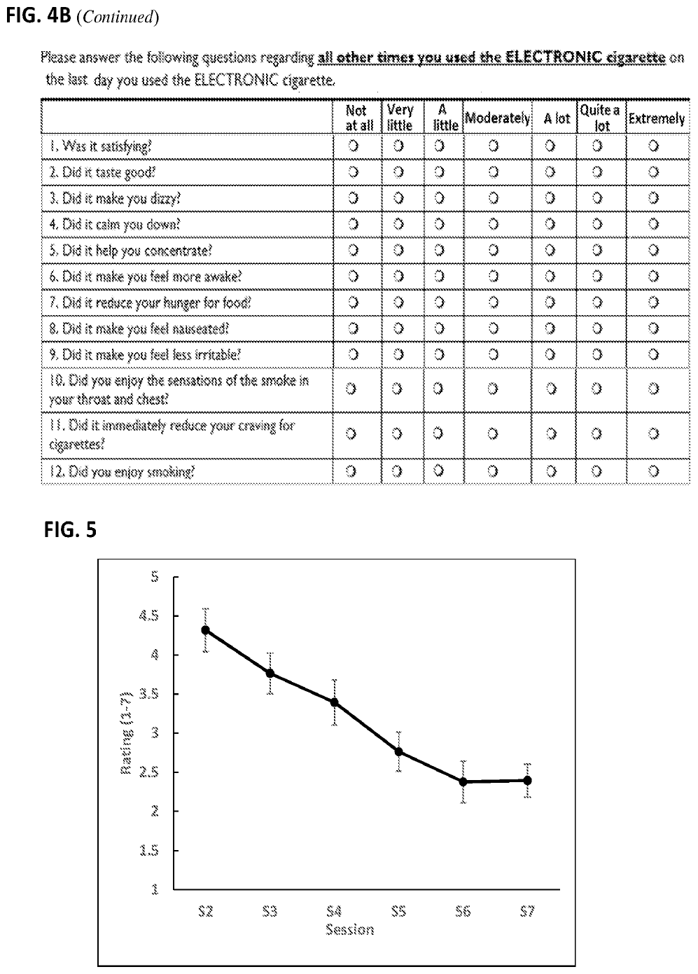

Modified Cigarette Evaluation Questionnaire-Extended (mCEQ-E) and Modified Electronic Cigarette Evaluation Questionnaire-Extended (mECEQ-E) The Cigarette Evaluation Questionnaire was initially developed in the PI's laboratory and used in numerous studies to assess the effects of pharmacological treatments on the rewarding effects of cigarette smoking. The mCEQ-E were utilized to assess the degree to which participants experience the reinforcing of smoking, providing five subscale scores computed as average per-item scores: smoking satisfaction (satisfying, tastes good, enjoy smoking), psychological rewards (calms down, more awake, less irritable, helps concentrate, reduces hunger), aversion (dizziness, nauseated), enjoyment of respiratory tract sensations (single-item assessment), craving reduction (single-item assessment). Participants were asked to assess the 12 items of the questionnaire on a 7-point scale, ranging from “not at all” to “extremely”. These 12 items were asked for the “first cigarette smoked”, “cigarette immediately after a meal”, and “all other cigarettes.” The e-cigarette version of this questionnaire (mECEQ-E) was also used. See FIGS. 4A and 4B.

Shiffman-Jarvik Withdrawal Scale The Shiffman-Jarvik Withdrawal Scale was used to measure withdrawal symptoms and a participant's desire to smoke. This scale consists of five subscales with average per-item scores computed: craving, psychological symptoms, physical symptoms, sedation, and appetite. (Lee Y Y, Khoo S, Morris T, et al. A mixed-method study of the efficacy of physical activity consultation as an adjunct to standard smoking cessation treatment among male smokers in Malaysia. SpringerPlus. 2016; 5(1): 2012. doi:10.1186/s40064-016-3675-2). The questionnaire of the Shiffman-Jarvik Withdrawal Scale is shown in FIG. 3.

The Fagerström Test for Nicotine Dependence The Fagerström Test for Nicotine Dependence is a six-item questionnaire developed by Karl-Olov Fagerström and is used to determine someone's level of nicotine dependence. The total scores obtained on the test allow the classification of nicotine dependence in three different levels: mild (0-3 points), moderate (4-6 points), and severe (7-10 points).

Participants who completed the study were contacted six months after the switch day utilizing an automated SMS messaging system, to ascertain their current smoking status and use of e-cigarettes. If a participant self-reported current abstinence from smoking they were asked to return to the office for collection of an expired air CO for verification.

All data measures (e.g., withdrawal symptoms questionnaires, smoking history, smoking diaries, etc.) were captured initially using paper or an electronic data capture system. Verified data files were analyzed using Statview or SAS (Statview, SAS Institute, Cary NC). Data was inspected for outliers and if sufficiently extreme (Chauvenet's criterion, after verifying normality of distributions) was censored from the data analysis.

### Outcomes

Switching Outcome Complete switching from combustible cigarette use at each time point was defined by a self-report of no cigarette smoking since the prior session, confirmed by an expired air CO reading of less than 5 ppm. The primary switching outcome was smoking abstinence during weeks 8-11 post-switching date. An intent-to-treat approach was taken in which any participants lost to follow-up after the point of randomization, or who smoked during weeks 8-11 were counted as having not completely switched to e-cigarette use.

A secondary outcome was 7-day point abstinence at 6 months post-switch, assessed by self-report utilizing an automated SMS messaging system. The main goal of the 6-month follow-up was to assess the persistence of switching to e-cigarettes.

Change in Rewarding Effects Change in reported rewarding effects of smoking for cigarettes smoked in the morning, after meals and at other times during the day was compared. Time Frame: First week compared to after start of study drugs. Characterize changes in the two primary scales of the Cigarette Evaluation Questionnaire (mCEQ), assessing smoking satisfaction and psychological reward, compared between cigarettes smoked after meals versus all others smoked during the day, examining how this difference changes after zonisamide/bupropion usage. The mCEQ uses a 7-point scale (0=Not at all; 1=Very little; 2=A little; 3=Moderately; 4=A lot, 5=Quite a lot; 6=Extremely) to measure the following subscales: Satisfaction, Psychological Reward, Enjoyment of Respiratory Tract Sensations, Craving Reduction, Aversion.

### Results

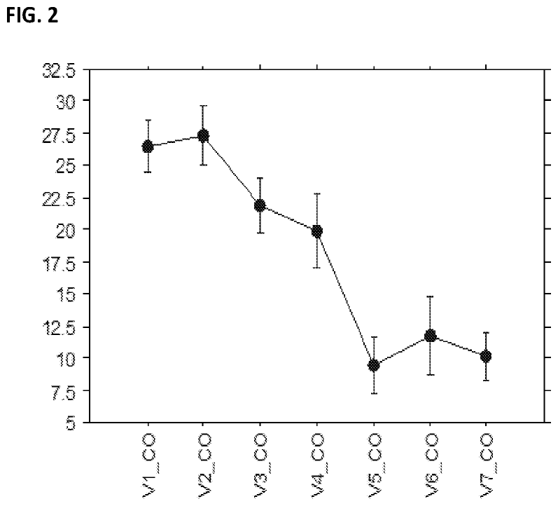

Expired CO Levels Success at giving up smoking was observed from this clinical study, see e.g., FIG. 2, which is a graph of the mean (±standard error) expired air carbon monoxide levels (CO) in ppm, a quantitative marker of cigarette smoke inhalation, for the study participants, during the 11 weeks after the target “switching” date, which would normally correspond to a “quit-smoking date.” Indeed, study participants were asked to quit smoking all combustible cigarettes when making the switch to the noncombustible e-cigarette alternative.

There was a roughly a 50% decrease in mean CO levels, indicating a marked reduction in smoking. Taking a conservative approach of imputing failure to participants who dropped out or were lost to contact, 50% (12 of 24) and 37.5% (9 of 24) achieved CO values less than 5 ppm at weeks 8 and 11, respectively, indicative of no or minimal smoking.

Abstinence If we impose a more stringent criterion, and require not only the expired air CO to be <5 ppm, but also a self-report of complete smoking abstinence (zero cigarettes smoked during the previous 4 weeks), then the abstinence rate was 45.8% at V6 (8 weeks after the target switch date) and 33.3% at V7 (at week 11). This is as high as the typical rate achieved with bupropion alone (˜25%), but what is notable is the absence of bupropion-associated side effects (see discussion below). This suggests that switching to an e-cigarette helped successful participants maintain their abstinence. There is also evidence that the pharmacotherapy in the present study added to the effects of the e-cigarettes, from a previous study (NCT04188197) conducted by us in the same region and drawing from the same population of smokers. In that prior study, which provided e-cigarettes without pharmacotherapy, the rate of complete abstinence from smoking was only 20% ( 10/50).

Side effects of and Adherence to Treatment. The treatment was well tolerated, with no dropouts attributed to medication side effects, which were generally mild (i.e., easily tolerated and not interfering with daily activities) and consistent with expected effects of zonisamide, bupropion and ENDS use. The most commonly reported side effects, all occurring at a rate of 17% ( 4/24), were throat irritation, constipation, nausea, and altered alertness. One of the nausea reports was moderate in intensity (i.e., interfering with daily activities), and was managed with a transient decrease in bupropion dosage. Another moderate rating of malaise occurred, which was attributed to inadvertently taking twice the scheduled dose of medications on that day. Only one incidence of (mild) insomnia occurred among the 25 study participants, whereas, according to the bupropion/Wellbutrin prescribing information, ˜20%, or 5 cases, would have been expected. Adherence to medication use was excellent; overall, participants reported taking the study medications on 95.2% of days (SD=16.96%). Counts of returned pills in unopened blister packs confirmed high adherence, with 95.6% (SD=7.1%) of pills taken.

ENDS use (in this Example, E-cigarettes use): During the last 4 weeks of the treatment period, 83.3% ( 20/24) of participants reported using ENDS, on 84.1% of days (SD=28.05). At 6 months, 32% ( 7/22) of all participants contacted, and 62.5% (⅝) of those who were abstinent at the end of treatment, reported using ENDS.

Smoking withdrawal symptoms: Withdrawal symptoms were minimal, with the exception of craving for cigarettes, which showed a marked decline over sessions, see FIG. 5.

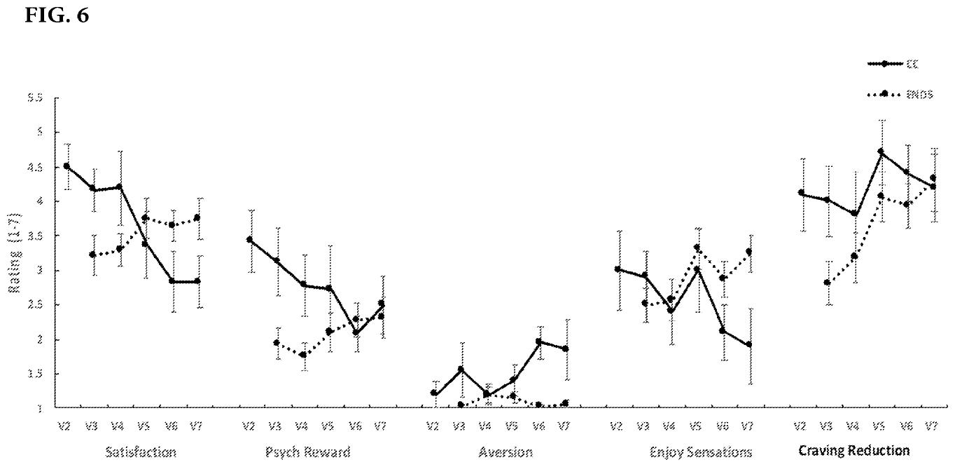

Rewarding/aversive effects: Ratings for the first Combustible Cigarette (“CC”) or ENDS (in this Example, E-cigarettes) of the day showed a very similar pattern of ratings as for the ones after a meal and all others. Therefore, only the ratings for the first use of the day are shown in FIG. 6. Satisfaction, psychological reward, and enjoyment of respiratory tract sensations for CC showed a pronounced decrease over time, while ratings of ENDS on all positive reward scales increased. In fact, reward ratings for ENDS exceeded those for CC by the end of treatment. Additionally, ratings of aversion for CC, in contrast to ratings for ENDS, increased over time.

### CONCLUSION

The above results show that the combination of bupropion, zonisamide, and e-cigarettes was effective in helping the subjects in giving up smoking cigarettes and switching from combustible cigarettes to e-cigarettes.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

All ranges recited herein also encompass any and all possible sub-ranges and combinations of sub-ranges thereof, as well as the individual values making up the range, particularly integer values. A recited range includes each specific value, integer, decimal, or identity within the range.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A method of treatment for promoting smoking cessation in a subject, the method comprising administering to the subject: a) bupropion or hydroxybupropion; b) an anticonvulsant or a GABAergic agent, and optionally c) a nicotine replacement or substitution product.

2. The method of claim 1, comprising administering to the subject a daily dose of bupropion ranging from about 150 mg to about 450 mg, e.g., about 300 mg to about 450 mg.

3. The method of claim 2, wherein the bupropion is administered in the form of a modified release formulation, such as a sustained release formulation or an extended release formulation, formulated for once daily or twice daily dosing.

4. The method of claim 2, wherein the bupropion is administered in the form of an immediate release formulation.

5. The method of any one of claims 2-4, wherein the bupropion is orally administered.

6. The method of any one of claims 2-5, wherein the bupropion is administered to the subject once daily or twice daily.

7. The method of any one of claims 2-6, wherein the bupropion is administered in the form of a pharmaceutically acceptable salt.

8. The method of any one of claims 2-7, comprising administering the bupropion to the subject in the morning.

9. The method of any one of claims 2-7, comprising administering the bupropion to the subject in the evening.

10. The method of any one of claims 1-9, wherein the anticonvulsant or GABAergic agent is zonisamide.

11. The method of claim 10, comprising administering zonisamide to the subject at a daily dose ranging from about 25 mg to about 400 mg, e.g., about 25 mg to about 100 mg, about 50 mg to about 100 mg, etc.

12. The method of claim 10 or 11, comprising administering zonisamide concurrently with bupropion, such as administering a dosage form comprising both zonisamide and bupropion as active ingredients, or concurrently administering separate dosage forms comprising zonisamide and bupropion, respectively.

13. The method of claim 10 or 11, comprising administering zonisamide and bupropion sequentially in any order.

14. The method of any one of claims 10-13, wherein the zonisamide is administered in an amount effective in reducing one or more adverse effects associated with bupropion or hydroxybupropion.

15. The method of any one of claims 10-14, wherein the zonisamide is administered in an amount effective in reducing incidences of insomnia and/or agitation without increasing incidences of somnolence or fatigue.

16. The method of any one of claims 10-14, wherein the zonisamide is administered in an amount effective in reducing incidences of seizure.

17. The method of any one of claims 10-16, wherein the zonisamide is administered in an amount effective in enhancing smoking cessation effects of bupropion.

18. The method of any one of claims 1-17, which does not administer to the subject a nicotine replacement or substitution product.

19. The method of any one of claims 1-17, comprising administering to the subject a nicotine replacement or substitution product.

20. The method of claim 19, wherein the nicotine replacement or substitution product is an e-cigarette, nicotine patch, nicotine gum, nicotine lozenge, nicotine pouch, nasal spray nicotine, dissolvable nicotine product, sublingual nicotine tablet, and/or nicotine inhaler (including nicotine salt inhaler, such as Philip Morris International's Platform 3 product).

21. The method of claim 19, wherein the nicotine replacement or substitution product is a modified risk tobacco product.

22. The method of claim 21, wherein the modified risk tobacco product is a smokeless tobacco such as chewable tobacco or a noncombustible tobacco product such as a heat-not-burn system (e.g., Philip Morris International's IQOS).

23. The method of any one of claims 1-22, wherein the subject is not in need of treatment of obesity.

24. The method of any one of claims 1-23, wherein the subject is a smoker who uses both e-cigarettes and combustible cigarettes prior to the treatment.

25. The method of any one of claims 1-24, wherein the subject smokes at least 10 commercially available cigarettes (combustible cigarettes) or equivalent per day for at least 12 months prior to the treatment.

26. The method of any one of claims 1-25, wherein the subject has an expired air CO reading of at least 10 ppm prior to the treatment.

27. The method of any one of claims 1-26, which reduces the subject's daily consumption of combustible cigarettes.

28. The method of any one of claims 1-27, which reduces the subject's expired air CO level by 20% or more (e.g., about 50%) compared to baseline and/or reduces the urinary NNAL (4-(methylnitrosamino)-1-(3-pyridyl)-1-butanol) by 20% or more (e.g., about 50%) compared to baseline.

29. The method of any one of claims 1-28, which reduces the subject's craving for combustible cigarettes, such as reduces the average per-item craving score of the subject to less than or equal to 4 on the 7 point Shiffman-Jarvik Withdrawal Scale (or less than 50 on a 100-point Visual Analog Scale) and/or the average per-item craving score of the subject does not increase by more than 1 point based on the 7 point Shiffman-Jarvik Withdrawal Scale (or by more than 10 points on a 100-point Visual Analog Scale).

30. The method of any one of claims 1-29, wherein the subject achieves smoking abstinence from combustible cigarettes.

31. A method of treatment for facilitating a smoker to switch from combustible cigarettes to e-cigarettes or another nicotine replacement or substitution product, the method comprising administering to the smoker: a) bupropion or hydroxybupropion; and b) an anticonvulsant or a GABAergic agent.

32. The method of claim 31, comprising administering to the smoker a daily dose of bupropion ranging from about 150 mg to about 450 mg, e.g., about 300 mg to about 450 mg.

33. The method of claim 32, wherein the bupropion is administered in the form of a modified release formulation, such as a sustained release formulation or an extended release formulation, formulated for once daily or twice daily dosing.

34. The method of claim 33, wherein the bupropion is administered in the form of an immediate release formulation.

35. The method of any one of claims 32-34, wherein the bupropion is orally administered.

36. The method of any one of claims 32-35, wherein the bupropion is administered to the smoker once daily or twice daily.

37. The method of any one of claims 32-36, wherein the bupropion is administered in the form of a pharmaceutically acceptable salt.

38. The method of any one of claims 32-37, comprising administering the bupropion to the smoker in the morning.

39. The method of any one of claims 32-38, comprising administering the bupropion to the smoker in the evening.

40. The method of any one of claims 31-39, wherein the anticonvulsant or GABAergic agent is zonisamide.

41. The method of claim 40, comprising administering zonisamide in a daily dose ranging from about 25 mg to about 400 mg, e.g., about 25 mg to about 100 mg, about 50 mg to about 100 mg, etc.

42. The method of claim 40 or 41, comprising administering zonisamide concurrently with bupropion, such as administering a dosage form comprising both zonisamide and bupropion as active ingredients, or concurrently administering separate dosage forms comprising zonisamide and bupropion, respectively.

43. The method of claim 40 or 41, comprising administering zonisamide and bupropion sequentially in any order.

44. The method of any one of claims 40-43, wherein the zonisamide is administered in an amount effective in reducing one or more adverse effects associated with bupropion or hydroxybuproprion.

45. The method of any one of claims 40-44, wherein the zonisamide is administered in an amount effective in reducing incidences of insomnia and/or agitation without increasing incidences of somnolence or fatigue.

46. The method of any one of claims 40-45, wherein the zonisamide is administered in an amount effective in reducing incidences of seizure.

47. The method of any one of claims 40-46, wherein the zonisamide is administered in an amount effective in enhancing smoking cessation effects of bupropion.

48. The method of any one of claims 31-47, for facilitating the smoker to switch from combustible cigarettes to e-cigarettes.

49. The method of any one of claims 31-47, for facilitating the smoker to switch from combustible cigarettes to a nicotine replacement or substitution product selected from an e-cigarette, nicotine patch, nicotine gum, nicotine lozenge, nicotine pouch, nasal spray nicotine, dissolvable nicotine product, sublingual nicotine tablet, nicotine inhaler (including nicotine salt inhaler, such as Philip Morris International's Platform 3 product), and a modified risk tobacco product, e.g., a smokeless tobacco such as chewable tobacco or a noncombustible tobacco product such as a heat-not-burn system (e.g., Philip Morris International's IQOS).

50. The method of any one of claims 31-49, wherein the smoker is not in need of treatment of obesity.

51. The method of any one of claims 31-50, wherein the smoker uses both e-cigarettes and combustible cigarettes prior to the treatment.

52. The method of any one of claims 31-51, wherein the smoker smokes at least 10 commercially available cigarettes (combustible cigarettes) or equivalent per day for at least 12 months prior to the treatment.

53. The method of any one of claims 31-52, wherein the smoker has an expired air CO reading of at least 10 ppm prior to the treatment.

54. The method of any one of claims 31-53, which reduces the smoker's daily consumption of combustible cigarettes.

55. The method of any one of claims 31-54, which reduces the smoker's expired air CO level by 20% or more (e.g., about 50%) compared to baseline and/or reduces the urinary NNAL (4-(methylnitrosamino)-1-(3-pyridyl)-1-butanol) by 20% or more (e.g., about 50%) compared to baseline.

56. The method of any one of claims 31-55, which reduces the smoker's craving for combustible cigarettes, such as reduces the average per-item craving score of the subject to less than or equal to 4 on the 7-point Shiffman-Jarvik Withdrawal Scale (or less than 50 on a 100-point Visual Analog Scale) and/or the average per-item craving score of the subject does not increase by more than 1 point based on the 7 point Shiffman-Jarvik Withdrawal Scale (or by more than 10 points on a 100-point Visual Analog Scale).

57. The method of any one of claims 31-56, wherein the smoker achieves smoking abstinence from combustible cigarettes.

58. A method of treatment for reducing a subject's craving for combustible cigarettes, comprising administering to the subject: a) bupropion or hydroxybupropion; b) an anticonvulsant or a GABAergic agent, and optionally c) a nicotine replacement or substitution product.

59. A method of treating dependency, addiction, or withdrawal associated with combustible cigarettes in a subject, the method comprising administering to the subject: a) bupropion or hydroxybupropion; b) an anticonvulsant or a GABAergic agent, and optionally c) a nicotine replacement or substitution product.

60. A kit comprising: a) one or more daily doses of bupropion, each comprising an extended release formulation comprising about 150 mg to about 450 mg bupropion; b) one or more daily doses of zonisamide, each comprising a pharmaceutical composition comprising about 25 mg to about 400 mg zonisamide, and optionally c) a nicotine replacement or substitution product.

61. The kit of claim 60, wherein the extended release formulation is formulated for once daily dosing.
